SEQUENCE LISTING (11 sequences)

SEQ ID NO: 1 - DNA, 2018 bp, artificial sequence
  synthetic oligonucleotide
gtgctgcggcgagctccgtccaaaagaaaatggggtttggtgtaaatctgggggtgtaatgttatcatatatcactctacctcgtaaaaccgacactgaaagctgccggacaacaaatcacaggtcaaaattatgagttcttcgtattatgtgaacgcgctttttagcaaatatacggcgggggcttctctgttccaaaatgccgagccgacttcttgctcctttgctcccaactcacagagaagcggctacggggcgggcgccggcgccttcgcctcgaccgttccgggcttatacaatgtcaacagccccctttatcagagcccctttgcgtccggctacggcctgggcgccgacgcctacggcaacctgccctgcgcctcctacgaccaaaacatccccgggctctgcagtgacctcgccaaaggcgcctgcgacaagacggacgagggcgcgctgcatggcgcggctgaggccaatttccgcatctacccctggatgcggtcttcaggacctgacaggaagcggggccgccagacctacacgcgctaccagacgctggagctggagaaggagttccacttcaaccgctacctgacgcggcgccgccgcattgaaatcgcccacgcgctctgcctcaccgagcgccagattaagatctggttccagaaccgccgcatgaagtggaagaaagagcataaggacgaaggtccgactgccgccgcagctcccgagggcgccgtgccctctgccgccgccactgctgccgcggacaaggccgacgaggaggacgatgatgaagaagaggaagacgaggaggaatgaggggccgatccggggccctctctgcaccggacagtcggaaaagcgtctttaagagactcactggttttacttacaaaaatgggaaaaataaaagaaaatgtaaaaaacaaaaacaaaaacaaaaaagcaacccagtccccaacctgcactctacccacccccatcacctactccagctcccaacttttgtggactgagcggccgcagagactgggtcgccttggattccctctgcctccgaggaccccaaaagacacccccaaccccaggccagccggccctgctctggcgcgtccaaaatactacctagcacaggcctctgctcgaggcacccccaaactacctatgtatccagccccagagggcctccattcccaggaagtccctatgtatcccaacactggcagacacccagcaccaccctcccagacccgcaagaaagtgaatctcactactacctactcccctaaaactacctattttgtgctggctggcttgcctgctacctagtgccgactgctcccaggcaagtcccctgctgcttacagcccgcagcttttggggtccctgaggctgccctgagaatgtgctgaggtccaggatcagggtattggcatctatttaaatcgaaaaataatatatttattccaaaaagcatcctaagtgcttgcaccctagaatcaatccctccttctctggcttggcacccacagctcaggcccatcaacccccacttctggaggggaatgttcctgagctggctgcagatctgtgggttagcttctgcttagcaggactgtggagatgcttccagcttcgctgtcctttcctctggctcctgtatcttactgttcagctgtgttaaatatgtacgccctgatgtttcctataatagcagatactgtatatttgaacaagatttttttttatcatttctatagtcttggagttcatttgtaaggcagtgtcttgacttggaaaggatgtgttaatggggtgactttgtagcatggtatgttgtcttgagttaactgtagtgggtggggaggtccaatgccctccgcaatgcccttcatctcctgtgttgtcctgtaccctgctcagctccatcctggggttcagggaaggcacacttcccagcccagctgtgttttatgtaaccgaaaataaagatgcgtggtgacaaagaaaaa

SEQ ID NO: 2 - DNA, 251 bp, artificial sequence
  synthetic oligonucleotide
ccggccctgctctggcgcgtccaaaatactacctagcacaggcctctgctcgaggcacccccaaactacctatgtatccagccccagagggcctccattcccaggaagtccctatgtatcccaacactggcagacacccagcaccaccctcccagacccgcaagaaagtgaatctcactactacctactcccctaaaactacctattttgtgctggctggcttgcctgctacctagtgccgactgctccca

SEQ ID NO: 3 - DNA, 178 bp, artificial sequence
  synthitic oligonucleotide
ccggccctgctctggcgcgtccaaaatactacctagcacaggcctctgctcgaggcacccccaaactaccttgtatccagcccgacccgcaagaaagtgaatctcactactacctctcccctaaaactacctttttgtgctggctggcttgcctgctacctagtgccgactgctccca

SEQ ID NO: 4 - DNA, 4152 bp, artificial sequence
  synthetic oligonucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgaccggccctgctctggcgcgtccaaaatactacctagcacaggcctctgctcgaggcacccccaaactaccttgtatccagcccgacccgcaagaaagtgaatctcactactacctctcccctaaaactacctttttgtgctggctggcttgcctgctacctagtgccgactgctcccagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 5 - DNA, 192 bp, artificial sequence
  synthetic oligonucleotide
cggccctgctctggcgcgtccaaaatactacctagcacaggcctctgctcgaggcacccccaaactaccttgtatccagcccgacccgcaagaaagtgaatctcactactacctctcccctaaaactacctttttgtgctggctggcttgcctgctacctagtgccgactgctcccagcggccgcggatcca

SEQ ID NO: 6 - DNA, 22 bp, artificial sequence
  synthetic oligonucleotide
cccaacaacatgaaactaccta

SEQ ID NO: 7 - DNA, 22 bp, artificial sequence
  synthetic oligonucleotide
cccaacaacatgaaactgccta

SEQ ID NO: 8 - DNA, 30 bp, artificial sequence
  synthetic oligonucleotide
tctcccaacaacatgaaactgcctattcac

SEQ ID NO: 9 - DNA, 137 bp, artificial sequence
  synthetic oligonucleotide
tcgactgctgttgaagtgagcgcctagcatgtttcatgttgatcggtagtgaagccacagatgtacccaacaacatgaaactacctagttgcctactgcctcggaagcttaataaaggatcttttattttcattggc

SEQ ID NO: 10 - DNA, 132 bp, artificial sequence
  synthetic oligonucleotide
tctcccaacaacatgaaactgcctattcacaattattctcccaacaacatgaaactgcctattcactctcccaacaacatgaaactgcctattcaccaatattctcccaacaacatgaaactgcctattcac

SEQ ID NO: 11 - RNA, 22 nt, artificial sequence
  synthetic oligonucleotide
ggguuguuguacuuugauggau

(19) United States
(12) Patent Application Publication
(10) Pub. No.: US 2013/0295160 A1
(43) Pub. Date: Nov. 7, 2013

(54) ANTAGONISTS OF MIR-196A

(71) Applicants: Baylor College of Medicine, Houston, TX (US); Gradalis, Inc., Carrollton, TX (US)

(72) Inventors: Donald Rao, Dallas, TX (US); Zhaohui Wang, Grapevine, TX (US); John J. Nemunaitis, Cedar Hill, TX (US); Changyi Chen, Houston, TX (US); Qizhi Yao, Houston, TX (US)

(21) Appl. No.: 13/738,742

(22) Filed: Jan. 10, 2013

Related U.S. Application Data
(60) Provisional application No. 61/585,092, filed on Jan. 10, 2012.

(51) Int. Cl. C12N 15/113; A61K 48/00

(52) U.S. Cl. CPC C12N 15/1135; A61K 48/005
USPC 424/450; 536/24.5; 514/44 R; 435/320.1

(57) ABSTRACT

A miR-196a antagonist capable of inhibiting a miR-196a activity, the miR-196a antagonist comprising one or more target sites for miR-196a. Included is also an expression vector comprising a promoter and a nucleic acid insert operably linked to the promoter, wherein the insert encodes one or more miR-196a antagonists capable of inhibiting a miR-196a activity. In one example, the one or more miR-196a antagonists comprise at least one stem-loop structure comprising a guide strand that comprises a sequence that is complementary to miR-196a, the stem-loop structure further comprising a passenger strand that comprises a mismatch.

### CROSS-REFERENCE TO RELATED APPLICATIONS

This application claims priority to U.S. Provisional Application Ser. No. 61/585,092, filed Jan. 10, 2012, the entire contents of which are incorporated herein by reference.

### TECHNICAL FIELD OF THE INVENTION

The present invention relates in general to inhibiting or antagonizing miR-196a activity as well as treating cancer. Examples of antagomir technology are provided. One application is the treatment of cancer, in particular, pancreatic ductal adenocarcinoma (PDAC).

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with antagomirs, cancer treatment, and miR-196a.

U.S. patent concerns methods and compositions for introducing miRNA activity or function into cells using synthetic nucleic acid molecules. Moreover, the U.S. patent concerns methods and compositions for identifying miRNAs with specific cellular functions that are relevant to therapeutic, diagnostic, and prognostic applications wherein synthetic miRNAs and/or miRNA inhibitors are used in library screening assays.

U.S. Patent Application Publication US 2007/0213292 A1 relates generally to chemically modified oligonucleotides useful for modulating expression of microRNAs and pre-microRNAs. More particularly, the application publication describes single stranded chemically modified oligonucleotides for inhibiting microRNA and pre-microRNA expression and to methods of making and using the modified oligonucleotides. Also described are compositions and methods for silencing microRNAs in the central nervous system.

U.S. Patent Application Publication 2009/0202493 describes methods of treating certain blood related disorders, in particular, thrombocytopenia and anemia comprising increasing miR-150 expression or inhibiting miR-150 in progenitor cells respectively.

U.S. Patent Application Publication 2010/0016406 provides a use of antisense RNA for the treatment, diagnosis and prophylaxis of cancer comprising administrating miRs 15 and 16 antisense RNA to a patient in need thereof.

### SUMMARY OF THE INVENTION

The present disclosures also provides for a miR-196a antagonist capable of inhibiting a miR-196a activity, the miR-196a antagonist comprising one or more target sites for miR-196a. The miR-196a antagonist may comprise of 1, 2, 3, 4, 5, 6, 7, 8, or 10 target sites for miR-196a. In another aspect, the miR-196a antagonist may comprise at least 11 target sites for miR-196a. In one aspect, the one or more target sites for miR-196a may comprise one or more HOXA7 target site for miR-196a. In other aspects, the one or more target sites for miR-196a may comprise at least five HOXA7 target site for miR-196a; one or more 3′ UTR of HOXB8 mRNA; one or more 3′ UTR of HOXB8 mRNA, wherein the one or more 3′ UTR of HOXB8 mRNA comprise at four miR-196a target sequences; at least 5 copies of 3′ UTR of HOXB8 mRNA; a sequence that is complementary to a mature miR-196a sequence; or at least one stem-loop structure comprising a guide strand that comprises a sequence that is complementary to miR-196a, the stem-loop structure further comprising a passenger strand that comprises a mismatch. In other aspects, the one or more target sites for miR-196a may comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof. Another embodiment includes an expression vector comprising a promoter and a nucleic acid insert operably linked to the promoter, wherein the insert encodes one or more miR-196a antagonists capable of inhibiting a miR-196a activity. The expression vector may be selected from the group consisting of viral vector, lentiviral vector, and plasmid. In one aspect, the vector backbone is miRZip or pUMVC3. The expression vector of claim 12, wherein the vector is in a bilamellar invaginated vesicle (BIV) liposomal delivery system. In one aspect, the vector is in a compacted DNA nanoparticle. The vector may also be compacted with one or more polycations that is a 10 kDA polyethylene glycol (PEG)-substituted cysteine-lysine 3-mer peptide (CK₃₀PEG10k). In one aspect, the vector is in a liposome comprising small molecule bivalent beta-turn mimics as receptor targeting moieties. The vector may comprise a miR-196a antagonist of 1, 2, 3, 4, 5, 6, 7, 8, or 10 target sites for miR-196a; in another aspect, the miR-196a antagonist may comprise at least 11 target sites for miR-196a. In one aspect, the vector may comprise one or more target sites for miR-196a that may comprise one or more HOXA7 target site for miR-196a, in other aspects, the one or more target sites for miR-196a may comprise at least five HOXA7 target site for miR-196a; one or more 3′ UTR of HOXB8 mRNA; one or more 3′ UTR of HOXB8 mRNA, wherein the one or more 3′ UTR of HOXB8 mRNA comprise at four miR-196a target sequences; at least 5 copies of 3′ UTR of HOXB8 mRNA; a sequence that is complementary to a mature miR-196a sequence; or at least one stem-loop structure comprising a guide strand that comprises a sequence that is complementary to miR-196a, the stem-loop structure further comprising a passenger strand that comprises a mismatch. In other aspects, the vector may comprise one or more target sites for miR-196a that may comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof. Another embodiment is a method for suppressing tumor cell growth, treating pancreatic ductal adenocarcinoma, or both in a human subject comprising the steps of identifying the human subject in need for suppression of the tumor cell growth, treatment of pancreatic ductal adenocarcinoma or both; and administering an expression vector in a therapeutic agent carrier complex to the human subject in an amount sufficient to suppress the tumor cell growth, treat pancreatic ductal adenocarcinoma or both, wherein the expression vector encodes one or more miR-196a antagonists capable of inhibiting a miR-196a activity in one or more target cells, wherein the inhibition results in suppressed tumor growth, a reduced tumor cell proliferation, or a reduced invasiveness of the tumor cells. In one aspect, the therapeutic agent carrier is a compacted DNA nanoparticle or a reversibly masked liposome decorated with one or more “smart” receptor targeting moieties that are small molecule bivalent beta-turn mimics. In one aspect, the therapeutic agent carrier is compacted DNA nanoparticles that are further encapsulated in a liposome. The therapeutic agent carrier may also be compacted DNA nanoparticle compacted with one or more polycations, wherein the one or more polycations is a 10 kDA polyethylene glycol (PEG)-substituted cysteine-lysine 3-mer peptide (CK30PEG10k) or a 30-mer lysine condensing peptide. The therapeutic agent carrier may also comprise reversibly masked liposome that are bilamellar invaginated vesicle (BIV). The vector may also be administered before, after, or concurrently as a combination therapy with one or more treatment methods selected from the group consisting of chemotherapy, radiation therapy, surgical intervention, antibody therapy, Vitamin therapy, or any combinations thereof. The expression vector may be selected from the group consisting of viral vector, lentiviral vector, and plasmid. In one aspect, the vector backbone is miRZip or pUMVC3. The expression vector of claim 12, wherein the vector is in a bilamellar invaginated vesicle (BIV) liposomal delivery system. In one aspect, the vector is in a compacted DNA nanoparticle. The vector may also be compacted with one or more polycations that is a 10 kDA polyethylene glycol (PEG)-substituted cysteine-lysine 3-mer peptide (CK₃₀PEG10k). In one aspect, the vector is in a liposome comprising small molecule bivalent beta-turn mimics as receptor targeting moieties. The vector may comprise a miR-196a antagonist of 1, 2, 3, 4, 5, 6, 7, 8, or 10 target sites for miR-196a; in another aspect, the miR-196a antagonist may comprise at least 11 target sites for miR-196a. In one aspect, the vector may comprise one or more target sites for miR-196a that may comprise one or more HOXA7 target site for miR-196a, in other aspects, the one or more target sites for miR-196a may comprise at least five HOXA7 target site for miR-196a; one or more 3′ UTR of HOXB8 mRNA; one or more 3′ UTR of HOXB8 mRNA, wherein the one or more 3′ UTR of HOXB8 mRNA comprise at four miR-196a target sequences; at least 5 copies of 3′ UTR of HOXB8 mRNA; a sequence that is complementary to a mature miR-196a sequence; or at least one stem-loop structure comprising a guide strand that comprises a sequence that is complementary to miR-196a, the stem-loop structure further comprising a passenger strand that comprises a mismatch. In other aspects, the vector may comprise one or more target sites for miR-196a that may comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof. Another embodiment is treating pancreatic ductal adenocarcinoma, or increasing effectiveness of a chemotherapeutic regimen to treat pancreatic ductal adenocarcinoma, or both in a human or animal subject, comprising the steps of identifying the human or animal subject suffering from pancreatic ductal adenocarcinoma or needing increased effectiveness of the chemotherapy against pancreatic ductal adenocarcinoma; and administering an expression vector in a therapeutic agent carrier complex to the human or animal subject in an amount sufficient to suppress or inhibit miR-196a activity in the human or the animal subject, wherein the expression vector expresses one or more miR-196a antagonists capable of inhibiting a miR-196a activity in one or more target cells in the human or animal subject, wherein the inhibition results in an enhanced action of the one or more chemotherapeutic agents, an arrested proliferation, reduced proliferation, or a reduced invasiveness of one or more tumor cells. In one aspect, the therapeutic agent carrier is a compacted DNA nanoparticle or a reversibly masked liposome decorated with one or more “smart” receptor targeting moieties that are small molecule bivalent beta-turn mimics. In one aspect, the therapeutic agent carrier is compacted DNA nanoparticles that are further encapsulated in a liposome. The therapeutic agent carrier may also be compacted DNA nanoparticle compacted with one or more polycations, wherein the one or more polycations is a 10 kDA polyethylene glycol (PEG)-substituted cysteine-lysine 3-mer peptide (CK30PEG10k) or a 30-mer lysine condensing peptide. The therapeutic agent carrier may also comprise reversibly masked liposome that are bilamellar invaginated vesicle (BIV). The vector may also be administered before, after, or concurrently as a combination therapy with one or more treatment methods selected from the group consisting of chemotherapy, radiation therapy, surgical intervention, antibody therapy, Vitamin therapy, or any combinations thereof. The expression vector may be selected from the group consisting of viral vector, lentiviral vector, and plasmid. In one aspect, the vector backbone is miRZip or pUMVC3. The expression vector of claim 12, wherein the vector is in a bilamellar invaginated vesicle (BIV) liposomal delivery system. In one aspect, the vector is in a compacted DNA nanoparticle. The vector may also be compacted with one or more polycations that is a 10 kDA polyethylene glycol (PEG)-substituted cysteine-lysine 3-mer peptide (CK₃₀PEG10k). In one aspect, the vector is in a liposome comprising small molecule bivalent beta-turn mimics as receptor targeting moieties. The vector may comprise a miR-196a antagonist of 1, 2, 3, 4, 5, 6, 7, 8, or 10 target sites for miR-196a; in another aspect, the miR-196a antagonist may comprise at least 11 target sites for miR-196a. In one aspect, the vector may comprise one or more target sites for miR-196a that may comprise one or more HOXA7 target site for miR-196a, in other aspects, the one or more target sites for miR-196a may comprise at least five HOXA7 target site for miR-196a; one or more 3′ UTR of HOXB8 mRNA; one or more 3′ UTR of HOXB8 mRNA, wherein the one or more 3′ UTR of HOXB8 mRNA comprise at four miR-196a target sequences; at least 5 copies of 3′ UTR of HOXB8 mRNA; a sequence that is complementary to a mature miR-196a sequence; or at least one stem-loop structure comprising a guide strand that comprises a sequence that is complementary to miR-196a, the stem-loop structure further comprising a passenger strand that comprises a mismatch. In other aspects, the vector may comprise one or more target sites for miR-196a that may comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C shows various proliferation assays. Stable miR-196a knockdown PC cell lines reduce cell proliferation. FIG. 1A. miR-196a levels were determined by real-time PCR between the stable miR-196a knockdown PC cell line PANC-1-zip-196a or AsPC-1-zip-196a and the corresponding control cell line PANC-1-zip-C or AsPC-1-zip-C. FIG. 1B. PANC-1-zip-196a and PANC-1-zip-C cells were seeded onto 96-well plates and treated with serum-free medium for 24 hours, and then cultured in the medium containing 1% serum. Cell proliferation was determined on days 0, 2, and 4 by MTT assay. Cell viability on day 0 was set as 100%. Values are the mean±SD of triplicate assays. FIG. 1C. AsPC-1-zip-196a and AsPC-1-zip-C cells were seeded onto 96-well plates and treated with serum-free medium for 24 hours, and then cultured in the medium containing 1% serum. Cell proliferation was determined on days 0, 2, and 4 by MTT assay. Cell viability on day 0 was set as 100%. Values are the mean±SD of triplicate assays.

FIGS. 2A and 2B show the effects of three miR-196a antagomirs on cellular miR-196a levels and cell proliferation in PANC-1 cells. FIG. 2A. PANC-1 cells were transfected with pUMVC3, pGBI-AS, pGBI-HA7, or pGBI-HB8 individually. After 48 hours, miR-196a levels were determined by real-time PCR. Values are the mean±SD of duplicate assays. *p<0.01 as compared to PANC-1 cells transfected with pUMVC3. FIG. 2B. PANC-1 cells were transfected with pUMVC3, pGBI-AS, pGBI-HA7, or pGBI-HB8 individually in a six-well plate. After 24 hours, cells were seeded onto 96-well plates, cultured in the medium containing 1% serum, and then cell proliferation was determined on days 0, 1, 3, and 5 by MTT assay. Cell viability on day 0 was set as 100%. Values are the mean±SD of triplicate assays. *p<0.01 as compared to PANC-1 cells transfected with pUMVC3.

FIGS. 3A to 3D show the Effects of miR-196a antagomir pGBI-HA7 on cell proliferation in PANC-1 and AsPC-1 cells. FIG. 3A. PANC-1 cells were transfected with pUMVC3 or pGBI-HA7 individually in a 6-well plate. After 24 hours, cells were seeded into 96-well plates, cultured in the medium containing 1% serum, and then cell proliferation was determined on days 0, 1, 3, and 5 by MTT assay. Cell viability on day 0 was set as 100%. Values are the mean±SD of triplicate assays. *p<0.01 as compared to PANC-1 cells transfected with pUMVC3. FIG. 3B. PANC-1 cells were transfected with pUMVC3 or pGBI-HA7 individually in a 6-well plate. After 24 hours, cells were seeded into 96-well plates, cultured in medium containing 5% serum, and then cell proliferation was determined on days 0, 1, 3, and 5 by MTT assay. Cell viability on day 0 was set as 100%. Values are the mean±SD of triplicate assays. *p<0.01 as compared to PANC-1 cells transfected with pUMVC3. FIG. 3C. AsPC-1 cells were transfected with pUMVC3 or pGBI-HA7 individually in a 6-well plate. After 24 hours, cells were seeded into 96-well plates, cultured in medium containing 1% serum, and then cell proliferation was determined on days 0, 1, 3, and 5 by MTT assay. Cell viability on day 0 was set as 100%. Values are the mean±SD of triplicate assays. *p<0.01 as compared to AsPC-1 cells transfected with pUMVC3. FIG. 3D. AsPC-1 cells were transfected with pUMVC3 or pGBI-HA7 individually in a 6-well plate. After 24 hours, cells were seeded into 96-well plates, cultured in medium containing 5% serum, and then cell proliferation was determined on days 0, 1, 3, and 5 by MTT assay. Cell viability on day 0 was set as 100%. Values are the mean±SD of triplicate assays. *p<0.01 as compared to AsPC-1 cells transfected with pUMVC3.

FIGS. 4A and 4B show the effects of miR-196a antagomir pGBI-HA7 on cell migration in PANC-1 and AsPC-1 cells. FIG. 4A. PANC-1 cells were transfected with pUMVC3 or pGBI-HA7 individually in a 6-well plate. After 24 hours, cells were seeded into the upper chamber of migration insert compartment. After incubation for another 24 hours, cell migration was determined using a modified Boyden chamber assay. Cell migration of PANC-1 cells transfected with pUMVC3 was set as 100%. Values are the mean±SD of triplicate assays. *p<0.01 as compared to PANC-1 cells transfected with pUMVC3. FIG. 4B. AsPC-1 cells were transfected with pUMVC3 or pGBI-HA7 individually in a 6-well plate. After 24 hours, cells were seeded into the upper chamber of migration insert compartment. After incubation for another 24 hours, cell migration was determined using a modified Boyden chamber assay. Cell migration of AsPC-1 cells transfected with pUMVC3 was set as 100%. Values are the mean±SD of triplicate assays. *p<0.01 as compared to AsPC-1 cells transfected with pUMVC3.

FIGS. 5A and 5B show the effects of miR-196a antagomir pGBI-HA7 on in vitro cell wound healing in PANC-1 and AsPC-1 cells. FIG. 5A. PANC-1 cells were transfected with pUMVC3 or pGBI-HA7 individually in a 6-well plate. Once >90% cell confluency (1 to 2 days after transfection) was attained, wounds were created in confluent monolayer cells by scratching cells with a sterile pipette tip. Wound healing was observed overtimes within the scrape lines. FIG. 5B. AsPC-1 cells were transfected with pUMVC3 or pGBI-HA7 individually in a 6-well plate. Once >90% cell confluency (1 to 2 days after transfection) was attained, wounds were created in confluent monolayer cells by scratching cells with a sterile pipette tip. Wound healing was observed overtimes within the scrape lines.

FIGS. 6A and 6C show the effects of miR-196a antagomir pGBI-HA7 on cell cycle progression and p27 expression in PANC-1 and AsPC-1 cells. FIG. 6A. PANC-1 cells were transfected with UMVC3 or pGBI-HA7 individually. After transfection 24 hours, cells were starved in serum-free culture medium for another 24 hours, then cells were given an stimulus with culture medium containing 2.5% serum for 24 hours again, and cell cycle was determined by flow cytometry after cells was stained with propidium iodide. Values are the mean±SD of triplicate assays. *p<0.05 and **p<0.01 as compared to PANC-1 cells transfected with pUMVC3. FIG. 6B. AsPC-1 cells were transfected with UMVC3 or pGBI-HA7 individually. After transfection 24 hours, cells were starved in serum-free culture medium for another 24 hours, then cells were given an stimulus with culture medium containing 2.5% serum for 24 hours again, and cell cycle was determined by flow cytometry after cells was stained with propidium iodide. Values are the mean±SD of triplicate assays. *p<0.05 and **p<0.01 as compared to AsPC-1 cells transfected with pUMVC3. FIG. 6C. PANC-1 or AsPC-1 cells were transfected with UMVC3 or pGBI-HA7 individually. After transfection for 48 hours, p27^{Kip1} protein expression was determined by western blot assay.

FIGS. 7A to 7B show the effect of pGBI-HA7 on subcutaneous tumor growth. FIG. 7A. The mice were euthanized at day 6 after the last treatment with DNA-lipoplexes pGBI-HA7 or pUMVC3 and tumor volumes were measured. Values are the mean of five mice±standard error. P<0.05. FIG. 7B. Immunohistochemistry staining of subcutaneous tumor with ki-67 (magnification of 100×).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Furthermore, the present application refers to certain vectors and data with nomenclature that varies that that filed originally. The following list of construct names correlates the previously filed application with the present disclosure (prior name->current name): pGBI-52->pGBI-AS, pGBI-53->pGBI-HA7, pGBI-54->pGBI-HB8. The sequences for the constructs and the data provided therewith are incorporated by reference in their entirety. Also, the new figures will have significant overlap with those of the prior filing, however, in certain instances more precise error bars and p-scores are provided herein.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “expression vector” as used herein in the specification and the claims includes nucleic acid molecules encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

The term “oncogene” as used herein refers to genes that permit the formation and survival of malignant neoplastic cells.

As used herein the term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The term “hybridizing” refers to any process by which a strand of nucleic acid binds with a complementary strand through base pairing.

The term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein the term “bi-functional” refers to a shRNA having two mechanistic pathways of action, that of the siRNA and that of the miRNA. A bifunctional construct concurrently repress the translation of the target mRNA (cleavage-independent, mRNA sequestration and degradation) and degrade (through RNase H-like cleavage) post-transcriptional mRNA through cleavage-dependent activities.

The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “liposome” refers to a closed structure composed of lipid bilayers surrounding an internal aqueous space. The term “polycation” as used herein denotes a material having multiple cationic moieties, such as quaternary ammonium radicals, in the same molecule and includes the free bases as well as the pharmaceutically-acceptable salts thereof.

Liposomal delivery system: The liposomal delivery system involves 1,2-dioleoyl-3-trimethyl-ammoniopropane (DOTAP) and cholesterol. This formulation combines with DNA to form complexes that encapsulate nucleic acids within bilamellar invaginated vesicles (liposomal BIVs). One of the inventors has optimized several features of the BIV delivery system for improved delivery of RNA, DNA, and RNAi plasmids. The liposomal BIVs are fusogenic, thereby bypassing endocytosis mediated DNA cell entry, which can lead to nucleic acid degradation and TLR mediated off-target effects.

The present inventors recognize that an optimized delivery vehicle needs to be a stealthed, which can achieved by PEGylation of nanoparticle with a zeta potential of ≦10 mV for efficient intravascular transport in order to minimize nonspecific binding to negatively-charged serum proteins such as serum albumin (opsonization). Incorporation of targeting moieties such as antibodies and their single chain derivatives (scFv), carbohydrates, or peptides may further enhance transgene localization to the target cell.

The present inventors have created targeted delivery of the complexes in vivo without the use of PEG thereby avoiding an excessively prolonged circulatory half-life. While PEGylation is relevant for DNA or siRNA oligonucleotide delivery to improve membrane permeability, the present inventors recognize that the approach may cause steric hindrance in the BIV liposomal structures, resulting in inefficient DNA encapsulation and reduced gene expression. Furthermore, PEGylated complexes enter the cell predominantly through the endocytic pathway, resulting in degradation of the bulk of the nucleic acid in the lysosomes. While PEG provides extremely long half-life in circulation, this has created problems for patients as exemplified by doxil, a PEGylated liposomal formulation that encapsulates the cytotoxic agent doxorubicin. Attempts to add ligands to doxil for delivery to specific cell surface receptors (e.g. HER2/neu) have not enhanced tumor-specific delivery.

The present disclosure includes embodiments in which BIVs are produced with DOTAP, and synthetic cholesterol using proprietary manual extrusion process. Furthermore, the delivery was optimized using reversible masking technology. Reversible masking utilizes small molecular weight lipids (about 500 Mol. Wt. and lower; e.g. n-dodecyl-β-D-maltopyranoside) that are uncharged and, thereby, loosely associated with the surface of BIV complexes, thereby temporarily shielding positively charged BIV complexes to bypass non-targeted organs. These small lipids are removed by shear force in the bloodstream. By the time they reach the target cell, charge is re-exposed (optimally ˜45 mV) to facilitate entry.

One reason that the BIV delivery system is uniquely efficient is because the complexes deliver therapeutics into cells by fusion with the cell membrane and avoid the endocytic pathway. The two major entry mechanisms of liposomal entry are via endocytosis or direct fusion with the cell membrane. The inventors found that nucleic acids encapsulated in BIV complexes delivered both in vitro and in vivo enter the cell by direct fusion and that the BIVs largely avoid endosomal uptake, as demonstrated in a comparative study with polyethylene-amine (PEI) in mouse alveolar macrophages. PEI is known to be rapidly and avidly taken up into endosomes, as demonstrated by the localization of >95% of rhodamine labeled oligonucleotides within 2-3 hrs post-transfection.

Cancer targeted delivery with decorated BIVs: The present inventors recognize that siRNAs that are delivered systemically by tumor-targeted nanoparticles (NPs) are significantly more effective in inhibiting the growth of subcutaneous tumors, as compared to undecorated NPs. Targeted delivery does not significantly impact pharmacokinetics or biodistribution, which remains largely an outcome of the EPR (enhanced permeability and retention) effect, but appears to improved transgene expression through enhanced cellular uptake.

Indeed, a key “missing piece” in development of BIVs for therapeutic is the identification of such non-immunogenic ligands that can be placed on the surface of BIV-complexes to direct them to target cells. While it might be possible to do this with small peptides that are multimerized on the surface of liposomes, these can generate immune responses after repeated injections. Other larger ligands including antibodies, antibody fragments, proteins, partial proteins, etc. are far more refractory than using small peptides for targeted delivery on the surface of liposomes. The complexes of the present invention are thus unique insofar as they not only penetrate tight barriers including tumor vasculature endothelial pores and the interstitial pressure gradient of solid tumors, but also target tumor cells directly. Therefore, the therapeutic approach of the present invention is not limited to delivery solely dependent on the EPR effect but targets the tumor directly.

Small molecules designed to bind proteins selectively can be used with the present invention. Importantly, the small molecules prepared are “bivalent” so they are particularly appropriate for binding cell surface receptors, and resemble secondary structure motifs found at hot-spots in protein-ligand interactions. The present inventors have adapted a strategy to give bivalent molecules that have hydrocarbon tails, and prepared functionalized BIV complexes from these adapted small molecules. An efficient high throughput technology to screen the library was developed and run.

Compacted DNA Nanoparticles: Safe and Efficient DNA Delivery in Post-Mitotic Cells: The Copernicus nucleic acid delivery technology is a non-viral synthetic and modular platform in which single molecules of DNA or siRNA are compacted with polycations to yield nanoparticles having the minimum possible volume. The polycations optimized for in vivo delivery is a 10 kDa polyethylene glycol (PEG) modified with a peptide comprising a N-terminus cysteine and 30 lysine residues (CK30PEG10k). The shape of these complexes is dependent in part on the lysine counterion at the time of DNA compaction. The minimum cross-sectional diameter of the rod nanoparticles is 8-11 nm irrespective of the size of the payload plasmid, whereas for ellipsoids the minimum diameter is 20-22 nm for typical expression plasmids. Importantly, these DNA nanoparticles are able to robustly transfect non-dividing cells in culture. Liposome mixtures of compacted DNA generate over 1.000-fold enhanced levels of gene expression compared to liposome naked DNA mixtures. Following in vivo dosing, compacted DNA robustly transfects post-mitotic cells in the lung, brain, and eye. In each of these systems the remarkable ability of compacted DNA to transfect post-mitotic cells appears to be due to the small size of these nanoparticles, which can cross the cross the 25 nm nuclear membrane pore.

One uptake mechanism for these DNA nanoparticles is based on binding to cell surface nucleolin (26 nm KD), with subsequent cytoplasmic trafficking via a non-degradative pathway into the nucleus, where the nanoparticles unravel releasing biologically active DNA. Long-term in vivo expression has been demonstrated for as long as 1 year post-gene transfer. These nanoparticles have a benign toxicity profile and do not stimulate toll-like receptors thereby avoiding toxic cytokine responses, even when the compacted DNA has hundreds of CpG islands and are mixed with liposomes, no toxic effect has been observed. DNA nanoparticles have been dosed in humans in a cystic fibrosis trial with encouraging results, with no adverse events attributed to the nanoparticles and with most patients demonstrating biological activity of the CFTR protein.

The present inventors recognize that expression of microRNA miR-196a is elevated in varieties of cancer and cancer cell lines. miR-196a is expressed from HOX gene clusters and tightly regulated. Dysregulation of miR-196a observed in cancer plays a critical role in cancer pathogenesis. Knockdown or antagonize miR-196a expression has significant clinical application for the treatment of cancer.

As non-limiting examples, three different expression constructs designed to antagonize the function of miR-196a in living cells are provided. All three constructs are designs to antagonize miR-196a action.

In one embodiment, an expression construct with single stem-loop structure in miR-30 backbone with the guide strand that contains sequences complementary to miR-196a sequence and the passenger strand with mis-matches is provided.

In further embodiment, an expression construct expressing a transcript that contains one or more HOXA7 target site for miR-196a is provided. In a preferred embodiment, the expression construct expressing a transcript contains five consecutive HOXA7 target site for miR-196a.

In a further embodiment, an expression construct expressing the 3′ UTR region of HOXB8 mRNA containing four predicted miR-196a target sequences are provided.

pGBI-HA7 (previously pGBI53): miR196a antagomir design: the human homeobox A7 (HOXA7) mRNA with accession number NM_{—}006896 (SEQ ID No: 1) contains five miR196a target sites at its 3′ untranslated region (3′ UTR) as emphasized by underlining below (SEQ ID No: 2):

(SEQ ID No: 1)
GTGCTGCGGCGAGCTCCGTCCAAAAGAAAATGGGGTTTGGTGTAAATCT
GGGGGTGTAATGTTATCATATATCACTCTACCTCGTAAAACCGACACTG
AAAGCTGCCGGACAACAAATCACAGGTCAAAATTATGAGTTCTTCGTAT
TATGTGAACGCGCTTTTTAGCAAATATACGGCGGGGGCTTCTCTGTTCC
AAAATGCCGAGCCGACTTCTTGCTCCTTTGCTCCCAACTCACAGAGAAG
CGGCTACGGGGCGGGCGCCGGCGCCTTCGCCTCGACCGTTCCGGGCTTA
TACAATGTCAACAGCCCCCTTTATCAGAGCCCCTTTGCGTCCGGCTACG
GCCTGGGCGCCGACGCCTACGGCAACCTGCCCTGCGCCTCCTACGACCA
AAACATCCCCGGGCTCTGCAGTGACCTCGCCAAAGGCGCCTGCGACAAG
ACGGACGAGGGCGCGCTGCATGGCGCGGCTGAGGCCAATTTCCGCATCT
ACCCCTGGATGCGGTCTTCAGGACCTGACAGGAAGCGGGGCCGCCAGAC
CTACACGCGCTACCAGACGCTGGAGCTGGAGAAGGAGTTCCACTTCAAC
CGCTACCTGACGCGGCGCCGCCGCATTGAAATCGCCCACGCGCTCTGCC
TCACCGAGCGCCAGATTAAGATCTGGTTCCAGAACCGCCGCATGAAGTG
GAAGAAAGAGCATAAGGACGAAGGTCCGACTGCCGCCGCAGCTCCCGAG
GGCGCCGTGCCCTCTGCCGCCGCCACTGCTGCCGCGGACAAGGCCGACG
AGGAGGACGATGATGAAGAAGAGGAAGACGAGGAGGAATGAGGGGCCGA
TCCGGGGCCCTCTCTGCACCGGACAGTCGGAAAAGCGTCTTTAAGAGAC
TCACTGGTTTTACTTACAAAAATGGGAAAAATAAAAGAAAATGTAAAAA
ACAAAAACAAAAACAAAAAAGCAACCCAGTCCCCAACCTGCACTCTACC
CACCCCCATCACCTACTCCAGCTCCCAACTTTTGTGGACTGAGCGGCCG
CAGAGACTGGGTCGCCTTGGATTCCCTCTGCCTCCGAGGACCCCAAAAG
ACACCCCCAACCCCAGGCCAGCCGGCCCTGCTCTGGCGCGTCCAAAATA
CTACCTAGCACAGGCCTCTGCTCGAGGCACCCCCAAACTACCTATGTAT
CCAGCCCCAGAGGGCCTCCATTCCCAGGAAGTCCCTATGTATCCCAACA
CTGGCAGACACCCAGCACCACCCTCCCAGACCCGCAAGAAAGTGAATCT
CACTACTACCTACTCCCCTAAAACTACCTATTTTGTGCTGGCTGGCTTG
CCTGCTACCTAGTGCCGACTGCTCCCAGGCAAGTCCCCTGCTGCTTACA
GCCCGCAGCTTTTGGGGTCCCTGAGGCTGCCCTGAGAATGTGCTGAGGT
CCAGGATCAGGGTATTGGCATCTATTTAAATCGAAAAATAATATATTTA
TTCCAAAAAGCATCCTAAGTGCTTGCACCCTAGAATCAATCCCTCCTTC
TCTGGCTTGGCACCCACAGCTCAGGCCCATCAACCCCCACTTCTGGAGG
GGAATGTTCCTGAGCTGGCTGCAGATCTGTGGGTTAGCTTCTGCTTAGC
AGGACTGTGGAGATGCTTCCAGCTTCGCTGTCCTTTCCTCTGGCTCCTG
TATCTTACTGTTCAGCTGTGTTAAATATGTACGCCCTGATGTTTCCTAT
AATAGCAGATACTGTATATTTGAACAAGATTTTTTTTTATCATTTCTAT
AGTCTTGGAGTTCATTTGTAAGGCAGTGTCTTGACTTGGAAAGGATGTG
TTAATGGGGTGACTTTGTAGCATGGTATGTTGTCTTGAGTTAACTGTAG
TGGGTGGGGAGGTCCAATGCCCTCCGCAATGCCCTTCATCTCCTGTGTT
GTCCTGTACCCTGCTCAGCTCCATCCTGGGGTTCAGGGAAGGCACACTT
CCCAGCCCAGCTGTGTTTTATGTAACCGAAAATAAAGATGCGTGGTGAC
AAAGAAAAA.

SEQ ID No: 2 is the following:

(SEQ ID No: 2)
CCGGCCCTGCTCTGGCGCGTCCAAAATACTACCTAGCACAGGCCTCTGC
TCGAGGCACCCCCAAACTACCTATGTATCCAGCCCCAGAGGGCCTCCAT
TCCCAGGAAGTCCCTATGTATCCCAACACTGGCAGACACCCAGCACCAC
CCTCCCAGACCCGCAAGAAAGTGAATCTCACTACTACCTACTCCCCTAA
AACTACCTATTTTGTGCTGGCTGGCTTGCCTGCTACCTAGTGCCGACTG
CTCCCA.

The present inventors can express this stretch of sequence to act as sponge to bind and reduce miR196a in transfected cells.

The miR196a target region was further modified and truncated (the strikethrough region). An A from an internal ATG was deleted to avoid translation of the antagomir. Excess sequences without miR196a site but with predicted target sites for other microRNA was deleted: CCGGCCCTGCTCTGGCGCGTCCAAAATACTACCTAGCACAGGCCTCTGCTCG AGGCACCCCCAAACTACCTTGTATCCAGCCC GACCCGCAAG AAAGTGAATCTCACTACTACCTACTCCCCTAAAACTACCTATTTTGTGCTGGCTGGC TTGCCTGCTACCTAGTGCCGACTGCTCCCA (SEQ ID No: 2).

The final sequence (SEQ ID No: 3), which can be inserted into pUMVC3 between Sal I and Not I sites is as follows (miR196a binding sequences are emphasized by underlining):

(SEQ ID No: 3)
CCGGCCCTGCTCTGGCGCGTCCAAAATACTACCTAGCACAGGCCTCTGC
TCGAGGCACCCCCAAACTACCTTGTATCCAGCCCGACCCGCAAGAAAGT
GAATCTCACTACTACCTCTCCCCTAAAACTACCTTTTTGTGCTGGCTGG
CTTGCCTGCTACCTAGTGCCGACTGCTCCCA.

The sequence of pGBI-HA7 (formerly pGBI 53) is the following (SEQ ID No: 4):

(SEQ ID No: 4)
tggccattgcatacgttgtatccatatcataatatgtacatttatattggctcatgtccaacattaccgccatgttgacattgattattga
ctagttattaatagtaatcaattacggggtcattagttcatagcccatatatggagttccgcgttacataacttacggtaaatggcccgcctggc
tgaccgcccaacgacccccgcccattgacgtcaataatgacgtatgttcccatagtaacgccaatagggactttccattgacgtcaatgggt
ggagtatttacggtaaactgcccacttggcagtacatcaagtgtatcatatgccaagtacgccccctattgacgtcaatgacggtaaatggcc
cgcctggcattatgcccagtacatgaccttatgggactttcctacttggcagtacatctacgtattagtcatcgctattaccatggtgatgcggtt
ttggcagtacatcaatgggcgtggatagcggtttgactcacggggatttccaagtctccaccccattgacgtcaatgggagtttgttttggca
ccaaaatcaacgggactttccaaaatgtcgtaacaactccgccccattgacgcaaatgggcggtaggcgtgtacggtgggaggtctatata
agcagagctcgtttagtgaaccgtcagatcgcctggagacgccatccacgctgttttgacctccatagaagacaccgggaccgatccagc
ctccgcggccgggaacggtgcattggaacgcggattccccgtgccaagagtgacgtaagtaccgcctatagactctataggcacacccct
ttggctcttatgcatgctatactgtttttggcttggggcctatacacccccgcttccttatgctataggtgatggtatagcttagcctataggtgtg
ggttattgaccattattgaccactccaacggtggagggcagtgtagtctgagcagtactcgttgctgccgcgcgcgccaccagacataata
gctgacagactaacagactgttcctttccatgggtcttttctgcagtcaccgtcgtcgacCGGCCCTGCTCTGGCGCGTCC
AAAATACTACCTAGCACAGGCCTCTGCTCGAGGCACCCCCAAACTACCTTGTATCC
AGCCCGACCCGCAAGAAAGTGAATCTCACTACTACCTCTCCCCTAAAACTACCTTTT
TGTGCTGGCTGGCTTGCCTGCTACCTAGTGCCGACTGCTCCCAGCGGCCGCGGATCC
Agatctttttccctctgccaaaaattatggggacatcatgaagccccttgagcatctgacttctggctaataaaggaaatttattttcattgcaat
agtgtgttggaattttttgtgtctctcactcggaaggacatatgggagggcaaatcatttaaaacatcagaatgagtatttggtttagagtttggc
aacatatgcccattcttccgcttcctcgctcactgactcgctgcgctcggtcgttcggctgcggcgagcggtatcagctcactcaaaggcgg
taatacggttatccacagaatcaggggataacgcaggaaagaacatgtgagcaaaaggccagcaaaaggccaggaaccgtaaaaagg
ccgcgttgctggcgtttttccataggctccgcccccctgacgagcatcacaaaaatcgacgctcaagtcagaggtggcgaaacccgacag
gactataaagataccaggcgtttccccctggaagctccctcgtgcgctctcctgttccgaccctgccgcttaccggatacctgtccgcctttct
cccttcgggaagcgtggcgctttctcatagctcacgctgtaggtatctcagttcggtgtaggtcgttcgctccaagctgggctgtgtgcacga
accccccgttcagcccgaccgctgcgccttatccggtaactatcgtcttgagtccaacccggtaagacacgacttatcgccactggcagca
gccactggtaacaggattagcagagcgaggtatgtaggcggtgctacagagttcttgaagtggtggcctaactacggctacactagaaga
acagtatttggtatctgcgctctgctgaagccagttaccttcggaaaaagagttggtagctcttgatccggcaaacaaaccaccgctggtag
cggtggtttttttgtttgcaagcagcagattacgcgcagaaaaaaaggatctcaagaagatcctttgatcttttctacggggtctgacgctcagt
ggaacgaaaactcacgttaagggattttggtcatgagattatcaaaaaggatcttcacctagatccttttaaattaaaaatgaagttttaaatcaa
tctaaagtatatatgagtaaacttggtctgacagttaccaatgcttaatcagtgaggcacctatctcagcgatctgtctatttcgttcatccatagt
tgcctgactcggggggggggggcgctgaggtctgcctcgtgaagaaggtgttgctgactcataccaggcctgaatcgccccatcatcca
gccagaaagtgagggagccacggttgatgagagctttgttgtaggtggaccagttggtgattttgaacttttgctttgccacggaacggtctg
cgttgtcgggaagatgcgtgatctgatccttcaactcagcaaaagttcgatttattcaacaaagccgccgtcccgtcaagtcagcgtaatgct
ctgccagtgttacaaccaattaaccaattctgattagaaaaactcatcgagcatcaaatgaaactgcaatttattcatatcaggattatcaatac
catatttttgaaaaagccgtttctgtaatgaaggagaaaactcaccgaggcagttccataggatggcaagatcctggtatcggtctgcgattc
cgactcgtccaacatcaatacaacctattaatttcccctcgtcaaaaataaggttatcaagtgagaaatcaccatgagtgacgactgaatccg
gtgagaatggcaaaagcttatgcatttctttccagacttgttcaacaggccagccattacgctcgtcatcaaaatcactcgcatcaaccaaac
cgttattcattcgtgattgcgcctgagcgagacgaaatacgcgatcgctgttaaaaggacaattacaaacaggaatcgaatgcaaccggcg
caggaacactgccagcgcatcaacaatattttcacctgaatcaggatattcttctaatacctggaatgctgttttcccggggatcgcagtggtg
agtaaccatgcatcatcaggagtacggataaaatgcttgatggtcggaagaggcataaattccgtcagccagtttagtctgaccatctcatct
gtaacatcattggcaacgctacctttgccatgtttcagaaacaactctggcgcatcgggcttcccatacaatcgatagattgtcgcacctgatt
gcccgacattatcgcgagcccatttatacccatataaatcagcatccatgttggaatttaatcgcggcctcgagcaagacgtttcccgttgaat
atggctcataacaccccttgtattactgtttatgtaagcagacagttttattgttcatgatgatatatttttatcttgtgcaatgtaacatcagagattt
tgagacacaacgtggctttccccccccccccattattgaagcatttatcagggttattgtctcatgagcggatacatatttgaatgtatttagaaa
aataaacaaataggggttccgcgcacatttccccgaaaagtgccacctgacgtctaagaaaccattattatcatgacattaacctataaaaat
aggcgtatcacgaggccctttcgtctcgcgcgtttcggtgatgacggtgaaaacctctgacacatgcagctcccggagacggtcacagctt
gtagtaagcggatgccgggagcagacaagcccgtcagggcgcgtcagcgggtgttggcgggtgtcggggctggcttaactatgcggc
atcagagcagattgtactgagagtgcaccatatgcggtgtgaaataccgcacagatgcgtaaggagaaaataccgcatcagattggctat

The antagomir sequences SEQ ID No: 5 (is underlined and in capital letters in representation of SEQ ID No: 4, above) is the following:

(SEQ ID No: 5)
CGGCCCTGCTCTGGCGCGTCCAAAATACTACCTAGCACAGGCCTCTGCT
CGAGGCACCCCCAAACTACCTTGTATCCAGCCCGACCCGCAAGAAAGTG
AATCTCACTACTACCTCTCCCCTAAAACTACCTTTTTGTGCTGGCTGGC
TTGCCTGCTACCTAGTGCCGACTGCTCCCAGCGGCCGCGGATCCA.

The insert sequence (single antagomir in mir-30 backbone) of pGBI-52 is the following (SEQ ID No: 9):

(SEQ ID No: 9)
TCGACTGCTGTTGAAGTGAGCGCCTAGCATGTTTCATGTTGATCGGTAG
TGAAGCCACAGATGTACCCAACAACATGAAACTACCTAGTTGCCTACTG
CCTCGGAAGCTTAATAAAGGATCTTTTATTTTCATTGGC

The insert sequence (four HOXB8 mRNA target site sequence) of pGBI-HB8 (formerly pGBI-54) is the following (SEQ ID No: 10):

(SEQ ID No: 10)
TCTCCCAACAACATGAAACTGCCTATTCACAATTATTCTCCCAACAACA
TGAAACTGCCTATTCACTCTCCCAACAACATGAAACTGCCTATTCACCA
ATATTCTCCCAACAACATGAAACTGCCTATTCAC

Pancreatic cancer (PC) is a devastating malignancy that represents the fourth leading cause of cancer-related death in the United States. According to a cancer statistics analyzed in 2010, the estimated new PC and death numbers in the United States in 2010 is 43140 and 36800, respectively (1). The overall survival duration of advanced PC patients is less than six months regardless of treatment. The poor outcome of PC is attributable mainly to late diagnosis and early metastasis of PC to other organs. Efficacy of current therapy for PC is limited (2). Therefore, developing new therapeutic strategies is urgently needed.

microRNAs (miRNAs) are a class of small noncoding RNAs that target multiple messenger RNAs by triggering translation repression and/or RNA degradation. The existence of miRNAs reveals a new mechanism of gene expression regulation and provides a new insight in cancer research. Extensive studies have strongly indicated highly diverse roles of miRNAs in cancer involved in cancer development, invasion, diagnosis, prognosis, and treatment. In fact, some miRNAs exert cancer-promoting effects mainly through the processes of either enhancing cancer cell proliferation and metastases or inhibiting apoptosis, while some miRNAs exhibit anti-cancer effects through the opposite effects (3, 4).

Recently, we demonstrated that miR-196a is overexpressed in PC. Specifically, in our study, randomly selected 10 PC cell lines all showed higher miR-196 levels than a non-cancerous human pancreatic ductal epithelial (HPDE) cell line, and 82% of pancreatic cancer tissues among 17 pairs of the tissue samples displayed increased miR-196a expression as compared with their adjacent non-cancerous pancreatic specimens (5). Functionally, miR-196a has been shown to have an oncogenic role in colorectal and esophageal cancers, as high levels of exogenous miR-196a promote migration, invasion, or the development of lung metastases of these cancer cells in mice (6-8).

It has been found that homeobox family genes play important roles in embryo development and some members of Homeobox family genes, including HOX-B8 and HOX-A7, are regulated by miR-196a, suggesting that miR-196a is also involved in embryonic development (9). In this study, for cancer therapeutic purpose, we have designed three plasmid-based miR-196a antagomirs, which express miR-196a anti-sense sequence (pGBI-AS), or predicted miR-196a target sequences at the 3′ untranslated regions (UTR) of HOX-A7 (pGBI-HA7) or HOX-B8 (pGBI-HB8). We hypothesized that the plasmid-based miR-196a antagomirs may bind to miR-196a, decreasing miR-196a levels or inhibiting its function as decoy inhibitors. The effects of these miR-196a antagomirs on pancreatic cancer cell proliferation and migration in vitro were determined.

1. Chemicals and reagents. Total RNA isolation reagents (mirVana™ miRNA Isolation Kit) were obtained from Ambion (Austin, Tex.). miRNA cDNA synthesis reagents (Mir-X miRNA First Strand Synthesis Kit) and real-time polymerase chain reaction (PCR) reagents (SYBR Advantage qPCR Premix) were purchased from Clontech (Mountain View, Calif.). Lentivector-based miR-196a knockdown construct and lentivirus packaging kits were obtained from System Biosciences (Mountain View, Calif.). X-tremeGENE HP DNA Transfection Reagent was obtained from Roche Applied Science (Indianapolis, Ind.). p27^{Kip1} and β-Actin antibodies were purchased from Cell Signaling Technology, Inc. (Danvers, Mass.).

2. Plasmid construct design and delivery system. As shown in Table 1, one completely anti-sense sequence of miR-196a, HOXA7 3′ UTR with five natural miR-196a target sites, and a five repeated miR-196a target sequence at 3′ UTR of HOX-B8 were cloned into pUMVC3 vector individually. The three plasmid constructs are named as pGBI-AS, pGBI-HA7, and pGBI-HB8, respectively. Plasmid transfection into PANC-1 or AsPC-1 cells was performed using X-tremeGENE HP DNA Transfection Reagent (Roche Applied Science) according to the manufacturer's protocol.

TABLE 1
Design of miR-196a antagomirs

| | | Sequence | |
|---|---|---|---|
| | | unit numbers | |
| | | cloning to | SEQ |
| Construct | Sequences used for | pUMVC 3 | ID |
| name | targeting miR-196a | vector | No.: |
| pGBI-AS* | Completely matched sequence | 1 | 6 |
| (formerly | to mature miR-196a: | | |
| pGBI-52) | CCCAACAACATGAAACTACCTA | | |
| pGBI- | HOXA7 3′ UTR with multiple | 1 | 3 |
| HA7** | miR-196a target site: | | |
| (formerly | CCGGCCCTGCTCTGGCGCGTCCAAAATAC | | |
| pGBI-53) | TACCTAGCACAGGCCTCTGCTCGAGGCAC | | |
| | CCCCAAACTACCTTGTATCCAGCCCGACC | | |
| | CGCAAGAAA | | |
| | GTGAATCTCACTACTACCTCTCCCCTAAA | | |
| | ACTACCTTTTTGTGCTGGCTGGCTTGCCTG | | |
| | CTACCTAGTGCCGACTGCTCCCA | | |
| pGBI- | HOXB8 with single 3′ UTR | 5 | 8 |
| HB8 | target site: | | |
| (formerly | TCTCCCAACAACATGAAACTGCCTATTCA | | |
| pGBI-54) | C | | |
| Mature | 5′-UAGGUAGUUUCAUGUUGUUGGG-3′ | | 11 |
| miR-196a | | | |
| Sequence | | | |

*The mature sequence of human miR-196a: TAGGTAGTTTCATGTTGTTGGG.
**Sequences underlined in HOXA7 3′ UTR with multiple miR-196a target site are the seed sequences for miR-196a binding.

3. Cell cultures. Human pancreatic cancer cell lines, PANC-1 and AsPC-1 were purchased from the American Type Culture Collection (Manassas, Va.). All cells were cultured as described (5).

4. miR-196a knockdown stable cell lines. miR-196a knockdown lentivirus was prepared following the manufacturer's protocol. Briefly, 293TN packaging cells were transfected with a lentivirus plasmid (miRZip control or miRZip-196a construct) with Lipofectamine 2000. 48 hours after transfection, supernatants containing viral particles were collected. Then PANC-1 or AsPC-1 cells were infected with the virus-containing supernatants and puromycin was added for miR-196a knockdown stable cell line selection.

4. Real-time PCR of miR-196a and ki-67. Total RNAs from PANC-1 or AsPC-1 cell lines transfected with different plasmid constructs and control vector pUMVC3 were isolated using mirVana™ miRNA Isolation Kit (Ambion) according to the manufacturer's instructions. Total RNA (0.5-1 μg) was converted into cDNA using Mir-X miRNA First Strand Synthesis Kit (Clontech) or iScript cDNA Synthesis Kit (Bio-Rad). miR-196a or ki-67 mRNA levels were determined by real-time PCR using SYBR Advantage qPCR Premix (Clontech). U6 RNA or GAPDH levels were used as loading controls. Real-time PCR amplification conditions were as follows: 10 minutes at 95° C., followed by 30 repeats of 15 seconds at 95° C. and 1 minute at 60° C. Cycle thresholds (Ct) were analyzed by iCycler iQ system from Bio-Rad laboratories (Hercules, Calif.).

Western Blot Analysis. Western blot analysis for p27^{Kip1} protein expression was performed as described previously (10).

6. Cell proliferation. Cell proliferation was analyzed with the MTT assay. PANC-1 or AsPC-1 cells transfected with different plasmids for 24 hours were seeded into 96-well plates at a density of 2,000 cells per well. At 24 hours after cells were seeded into 96-well plates, cell culture medium was replaced by fresh medium containing different serum concentrations ranging from 1-5%. Cell growth was assessed on days 0, 1, 3, and 5. Absorbance was recorded at 490 nm with an EL-800 universal microplate reader (Bio-Tek Instruments, Winooski, Vt.). For cell proliferation of miR-196a knockdown stable PANC-1 or AsPC-1 cell line (PANC-1-zip-196a or AsPC-1-zip-196a) and its control cell lines (PANC-1-zip-C or AsPC-1-zip-C), PANC-1-zip-196 a and PANC-1-zip-C cells or AsP C-1-zip-196 a and AsP C-1-zip-C cells were seeded directly into 96-well plates at a density of 2,000 cells per well, and cells were starvated for 24 hours in serum-free medium. Then, cells were treated with medium containing different serum concentrations ranging from 1-5%.

7. Cell migration. Cell migration was determined using a modified Boyden chamber assay. At 24 hours after PANC-1 or AsPC-1 cells transfected with different plasmids, Cells were trypsinized and resuspended in growth medium (10⁵ cells/200 μl) were added into the upper chamber of migration insert compartment and 600 μl of the same growth medium was added into the lower chamber. After 24 hours cells were incubated in 4 μM Calcein-AM (Molecular Probes, Eugene, Oreg.) for 1 hr at 37° C., and then cells were fixed with 4% paraformaldehyde. The fluorescence was read from the bottom at an excitation wavelength of 495 nm and emission wavelength of 520 nm. Cells in the upper chamber were then removed, and cells that had migrated onto the lower surface of the membrane were quantified. The migration/invasion rate was presented as the ratio of the mean fluorescence reading after scraping of the cells divided by the reading before removing the top cells.

8. Wound healing assay. A monolayer wound healing assay was also performed. Cells were seeded onto 6-well plates in growth medium. Once >90% confluency was attained, wounds were created in confluent monolayer cells by scratching cells with a sterile pipette tip. Wound healing was observed overtimes within the scrape lines. Representative fields for wound healing were photographed.

9. Flow Cytometry. Cells were trypsinized, washed once with cold PBS, and then fixed with 70% ethanol overnight at 4° C. Fixed cells were suspended in PBS containing 25 μg/mL propidium iodide (Roche Diagnostics, Indianapolis, Ind.) and 10 μg/mL RNase A (Sigma-Aldrich, St. Louis, Mo.) at 37° C. for 30 minutes. Flow cytometry analysis for cell cycle distribution was performed as previously described (10).

10. in vivo animal study. The formulation of Either pUMVC3 or pGBI-HA7 with DOTAP:Chol liposomes was performed as previously described. AsPC-1 cells (1.5×10⁶) were subcutaneously injected into the right flank region of the body of 5- to 6-week-old male nude mice (NCI-Charles River). All mice were cared for in accordance with an animal protocol approved by Baylor College of Medicine Institutional Animal Care and Use Committee (IACUC). After 10 days' inoculation of subcutaneous AsPC-1 cell tumor, the mice were divided into two groups randomly and there were five mice in each group. The treatment was performed as follows: 100 uL (30 ug) of pUMVC3 and pGBI-HA7 DNA-lipoplexes were introduced into systemic circulation of control group and treatment group mice, respectively, via tail vein injection. The treatment was performed every 4 days and totally six injections were done. The tumor size was measured every four days by using a digital caliper (VWR International), and the tumor volume was determined with the formula: tumor volume [mm³]=(length [mm])×(width [mm])²×0.52. The mice were euthanized 6 days after the last treatment.

11. Immunohistochemistry. Subcutaneous tumor samples removed from mice were fixed in 10% formalin over night, and washed with water and transferred to 70% ethanol. The samples then were embedded in paraffin, sectioned to 5 μM thickness, and stained with anti-ki-67 (1:100 dilution). Counterstaining was performed by staining the samples with hematoxylin.

12. Statistical analysis. Data from real-time PCR, MTS, migration, and mirgration assay were expressed as mean±SEM. Significant differences were determined by Student's t-test (p<0.05).

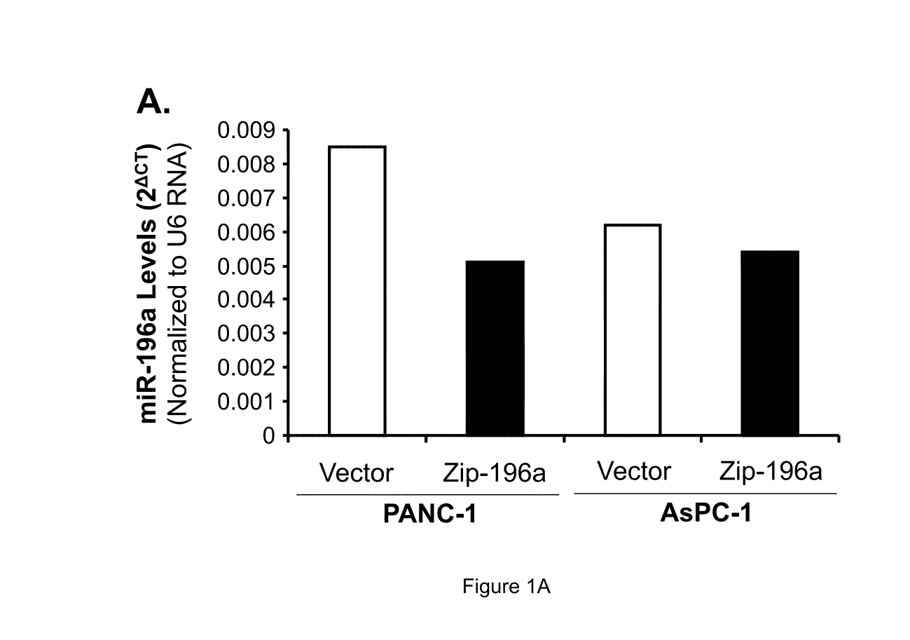

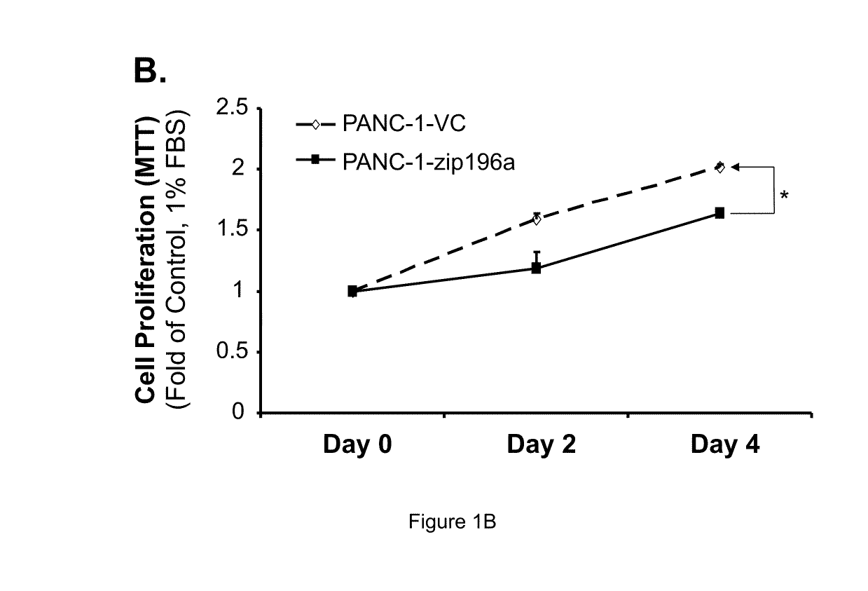

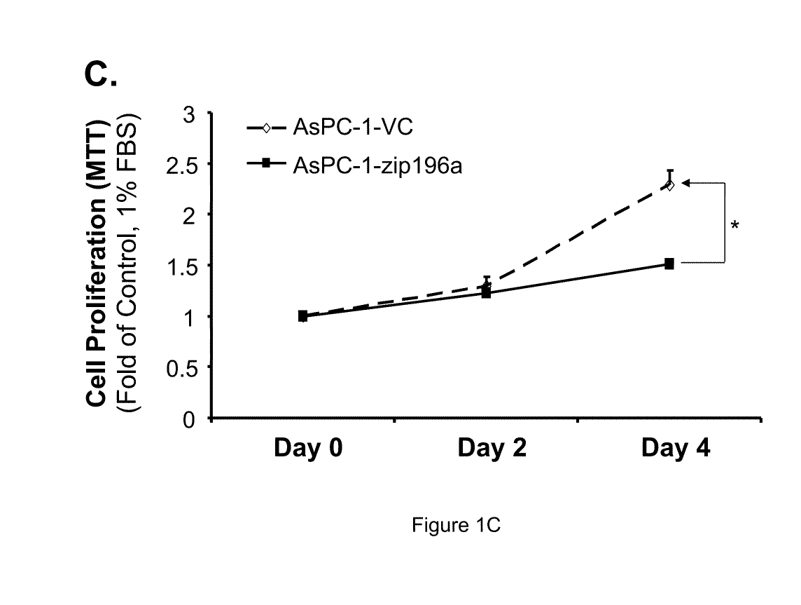

Lentiviral vector-mediated stable knockdown of miR-196a levels inhibits cell proliferation in human pancreatic cancer cell lines. We first determined whether miR-196a has an oncogenic role on PC. To this end, we established two stable miR-196a knockdown cell lines from PANC-1 and AsPC-1 cells by lentivirus-mediated gene transfer system. The miR-196a knockdown cell lines from PANC-1 and AspC-1 cells are named PANC-1-zip-196a and AsPC-1-zip-196a respectively, while the corresponding control cell lines also are established and are named PANC-1-zip-C and AsPC-1-zip-C. As shown in FIG. 1A, PANC-1-zip-196a and AsPC-1-zip-196a cells showed 10% to 30% reduction in miR-196a levels compared with the control cells. And in the subsequent cell proliferation study (FIG. 1B, 1C), both PANC-1-zip-196a and AsPC-1-zip-196a cells dysplayed reduced cell growth with respect to the control cells. These results demonstrate that miR-196a has an oncogenic property that increases cell proliferation in pancreas originated cancer cell lines PANC-1 and AsPC-1, thus, we have developed a robust targeted therapy for PC by targeting miR-196a.

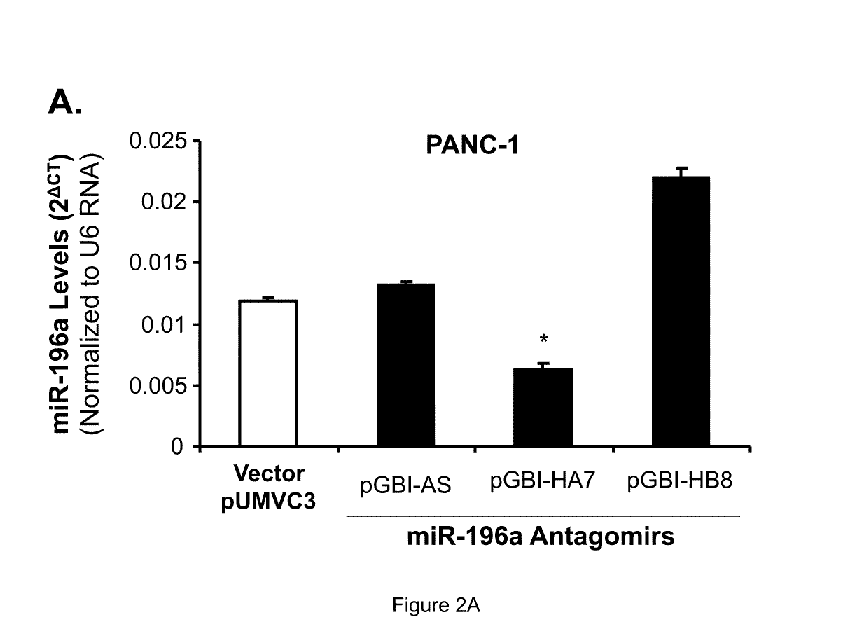

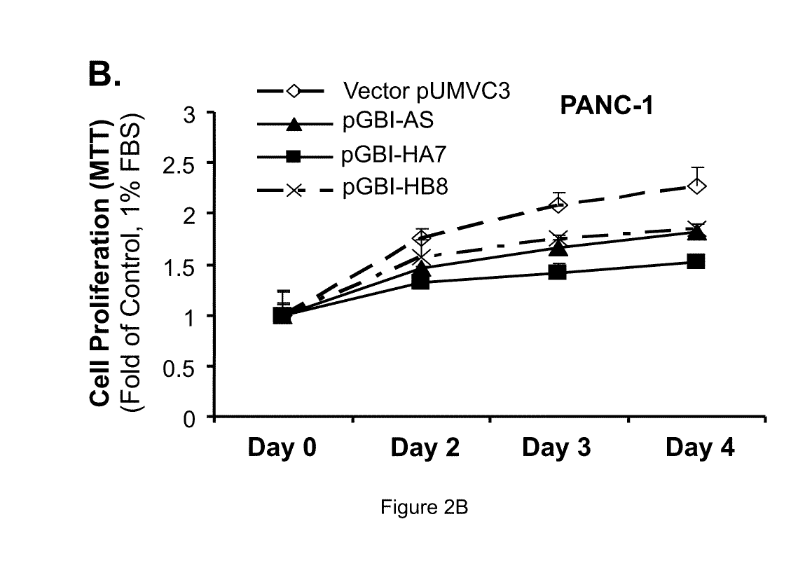

miR-196a antagomir pGBI-HA7 reduces miR-196a levels and inhibits cell proliferation in human pancreatic cancer cell lines. To determine whether these three plasmid constructs (pGBI-AS, pGBI-HA7, and pGBI-HB8) could knockdown miR-196a expression levels, we transfected each of these three constructs and a control plasmid pUMVC3 into a PC line PANC-1, which exhibits a high miR-196a expression level. After the transfection for 48 hours, miR-196a levels were determined by real-time PCR assay. As shown in FIG. 2A, compared with the control plasmid pUMVC3, pGBI-HA7 transfection resulted in proximately 50% reduction of miR-196a levels. However, pGBI-AS did not change miR-196a expression significantly and pGBI-HB8 even increased miR-196a levels.

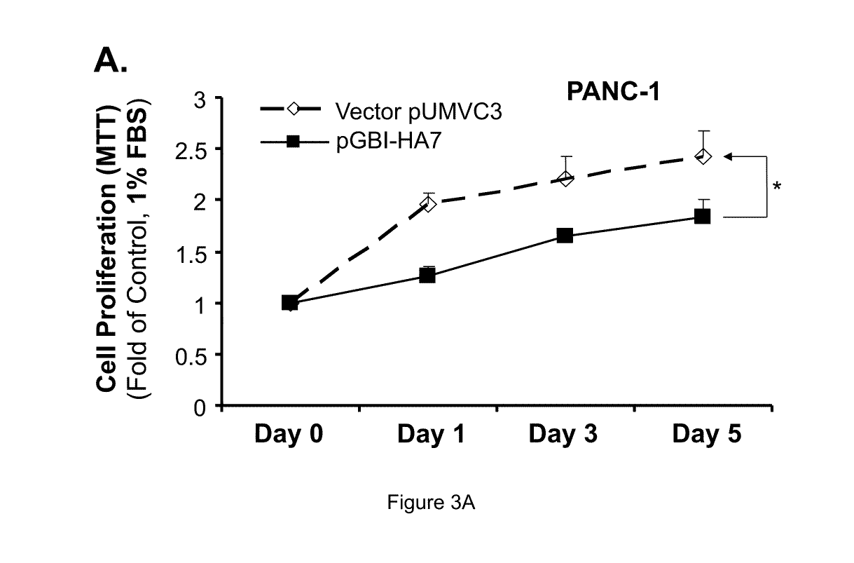

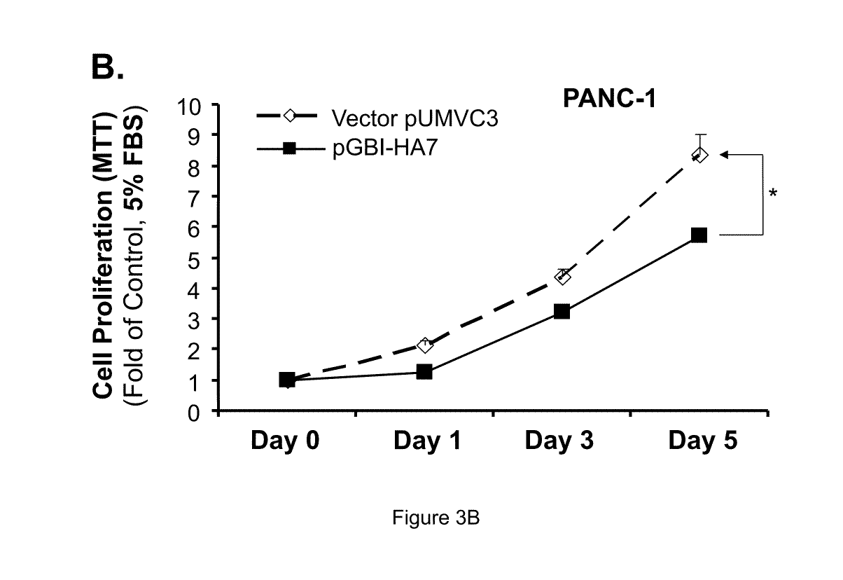

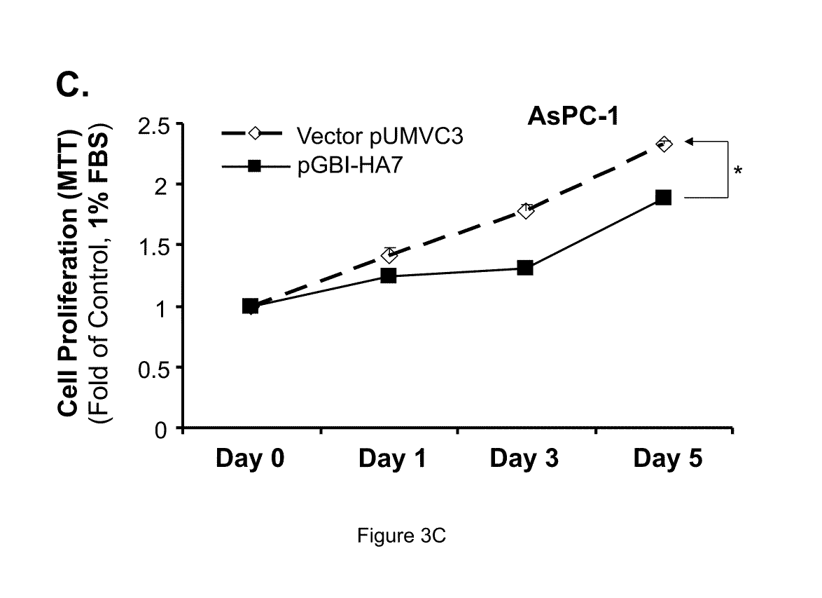

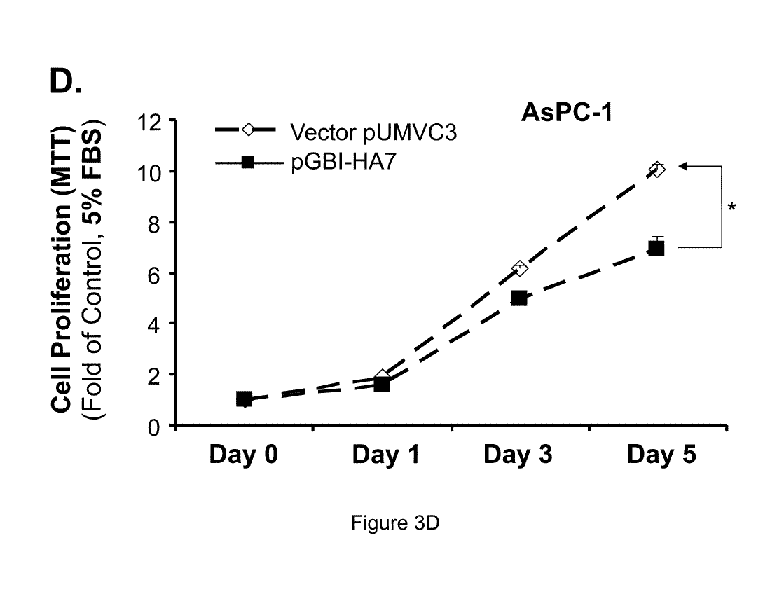

In order to examine whether these three plasmid constructs exert any functions on PC progression, we measured cell proliferation using MTT assay. All three plasmids reduced PANC-1 cell proliferation under the condition of cell culture medium containing 1% serum on days 1, 3, or 5 after their transfection into the cells, indicating that all these three constructs may have an effect on inhibiting miR-196a growth (FIG. 2B). Notably, pGBI-HA7 exhibited a more significant inhibition on PANC-1 cell proliferation than the other two plasmids pGBI-AS and pGBI-HX8 (FIG. 2B) did. Thus, these data suggest that pGBI-HA7 is the best construct of three plasmids tested, and we focused on pGBI-HA7 for further investations. In a subsequent study, we demonstrated that pGBI-HA7 reduced PANC-1 cell proliferation not only under the condition of culture medium with 1% serum (FIG. 3A) but also under culture medium containing 5% serum (FIG. 3B). Next, we extended our proliferation study to another PC cell line AsPC-1, which also expresses higher miR-196a. Like PANC-1 cells, AsPC-1 cells also showed reduced cell proliferation after pGBI-HA7 transfection under cell culture condition containing 1% or 5% serum (FIGS. 3C and 3D).

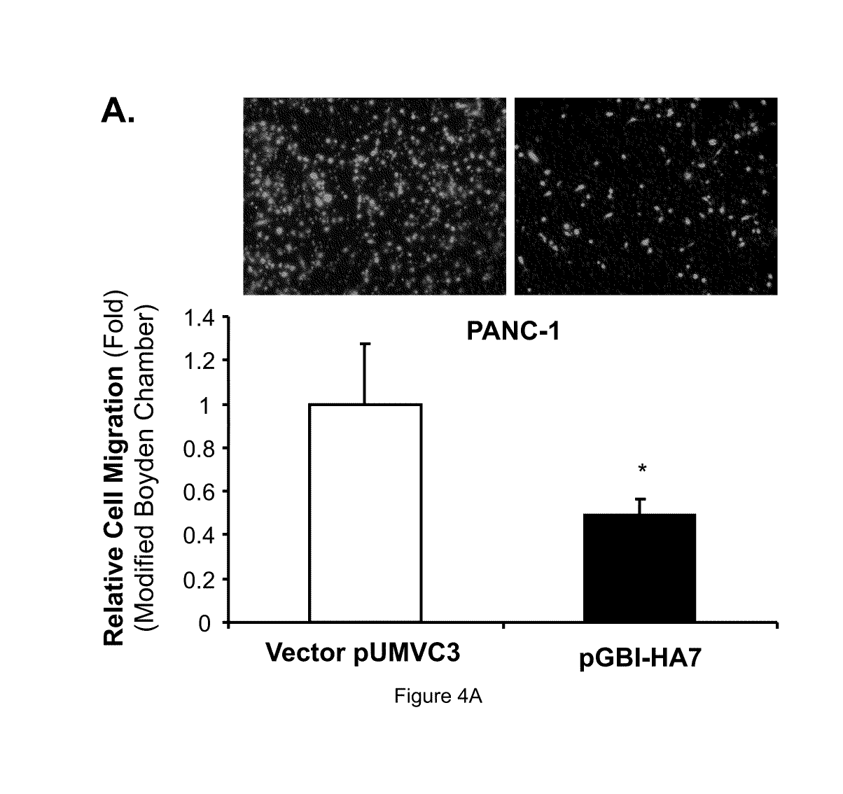

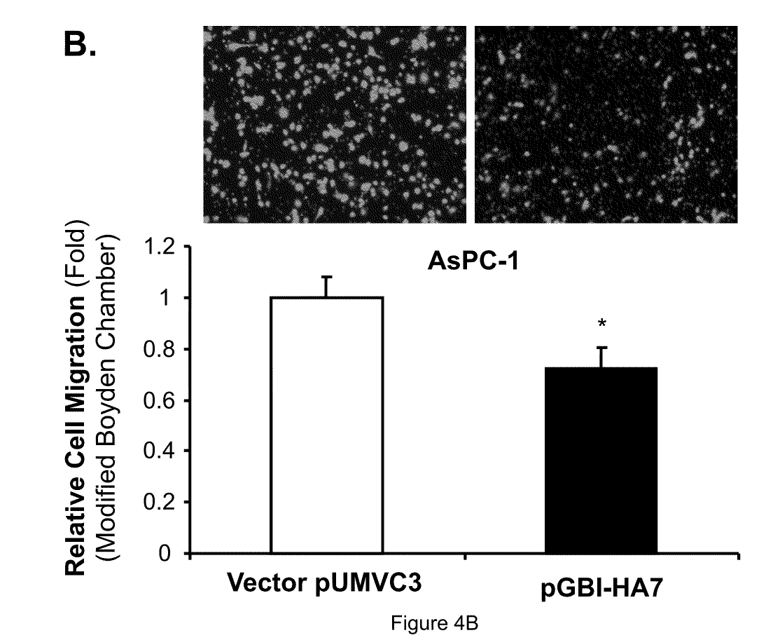

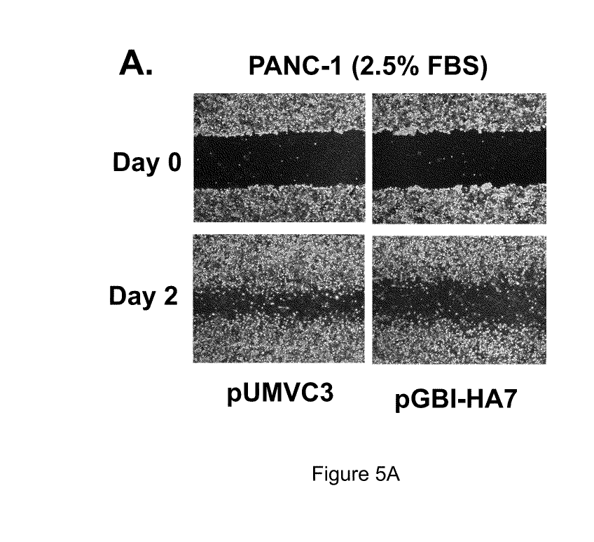

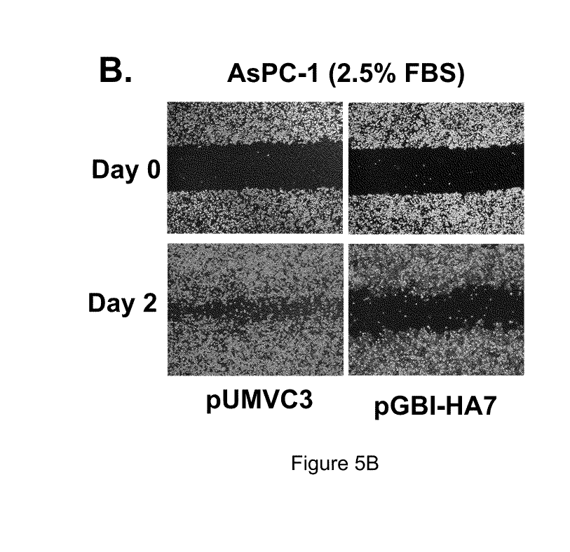

miR-196a antagomir pGBI-HA7 inhibits cell migration in human pancreatic cancer cell lines. In addition to reducing cell proliferation, we were interested in whether miR-196a antagomir pGBI-HA7 could inhibit PC cell migration in vitro. Cell migration was determined using a modified Boyden chamber assay. As shown in FIGS. 4A and 4B, both PANC-1 and AsPC-1 cells showed reduced migration after pGBI-HA7 transfection. In support to these results, wound-healing assay also demonstrated delayed wound healing in both PANC-1 and AsPC-1 cells transfected with pGBI-HA7 (FIGS. 5A and 5B). These results suggest that miR-196a antagomir pGBI-HA7 might be a potential therapeutics for reducing metastasis in vivo.

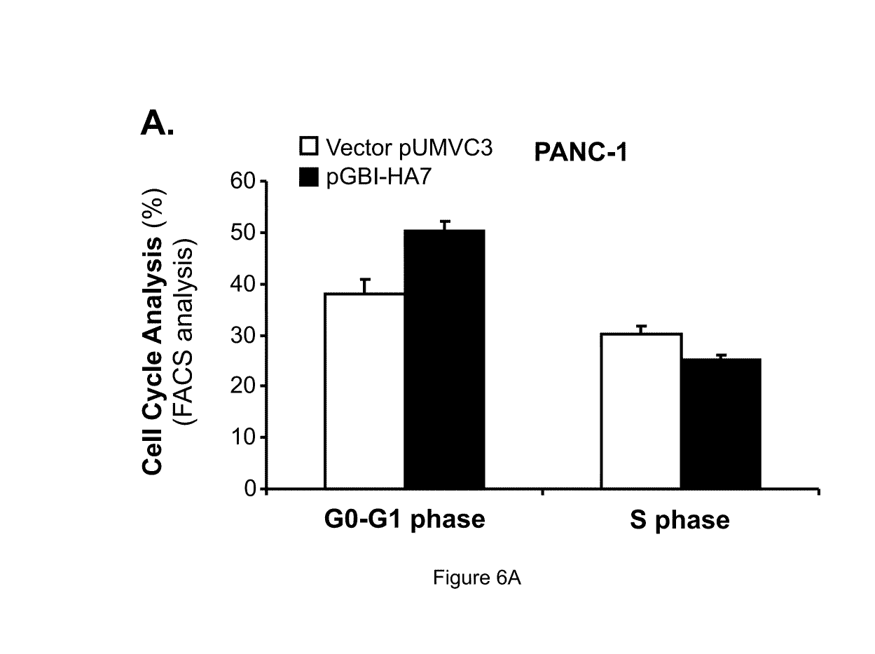

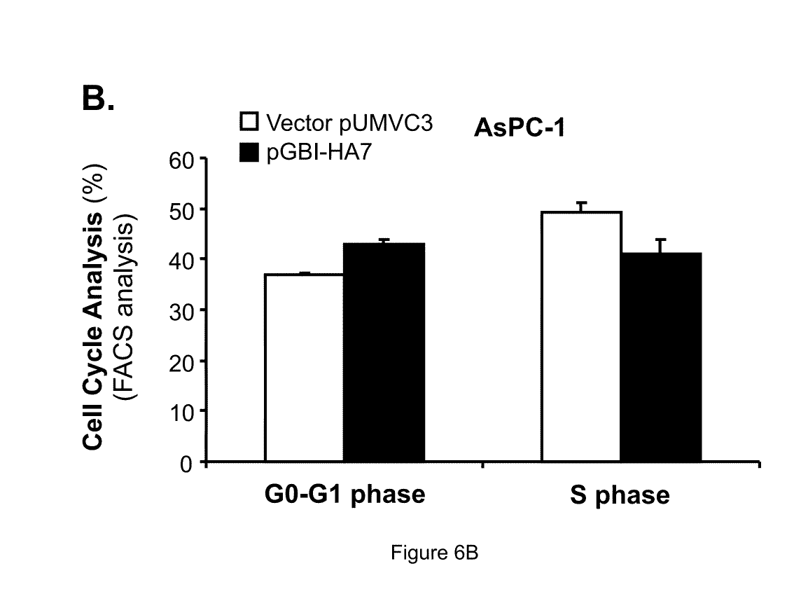

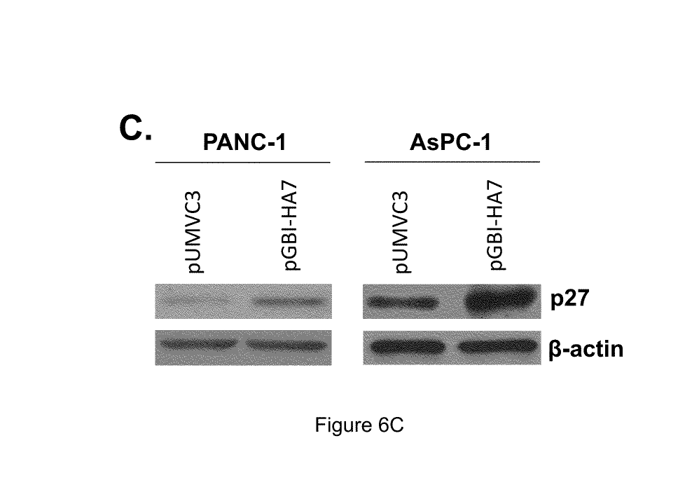

miR-196a antagomir pGBI-HA7 inhibits cell cycle progression in human pancreatic cancer cell lines. Since miR-196a antagomir pGBI-HA7 reduced cell proliferation in PANC-1 and AsPC-1 cell lines, we examined cell cycle changes after pGBI-HA7 transfection. For this purpose, PANC-1 or AsPC-1 cells were starved in serum-free culture medium for 24 hours after pGBI-HA7 transfection, then the cells were given a stimulus with culture medium containing 5% serum for another 24 hours, and finally, cell cycles were determined by flow cytometry after propidium iodide staining. The results showed that pGBI-HA7 induced more cell arrests at G1/G0 phase and fewer cell arrests at S phase (FIGS. 6A and 6B). These results are consistent with the inhibitory effect of cell proliferation induced by pGBI-HA7.

p27^{Kip1} (CDKN1B) gene encodes an enzyme which belongs to the Cip/Kip family of cyclin dependent kinase (Cdk) inhibitor proteins. p27^{Kip1} binds to and prevents the activation of cyclin E-CDK2 or cyclin D-CDK4 complexes, and thus controls the cell cycle progression at G1. p27^{Kip1} is believed to act as a cell cycle inhibitor as it can make cells stop at G1 phase of the cell cycle, thus slowing down the cell division (11). Since p27^{Kip1} has critical functions on the cell cycle regulation, and also it is a predicted miR-196a target gene, we investigated whether miR-196a antagomir pGBI-HA7 could regulate p27^{Kip1} expression. As shown in FIG. 6C, pGBI-HA7 transfection increased p27^{Kip1} protein levels in both PANC-1 and AsPC-1 cell lines. As p27^{Kip1} can block cell cycle progression at G1 phase, pGBI-HA7-reduced proliferation in PANC-1 or AsPC-1 cells could be explained at least partially by pGBI-HA7-mediated p27^{Kip1} protein upregulation.

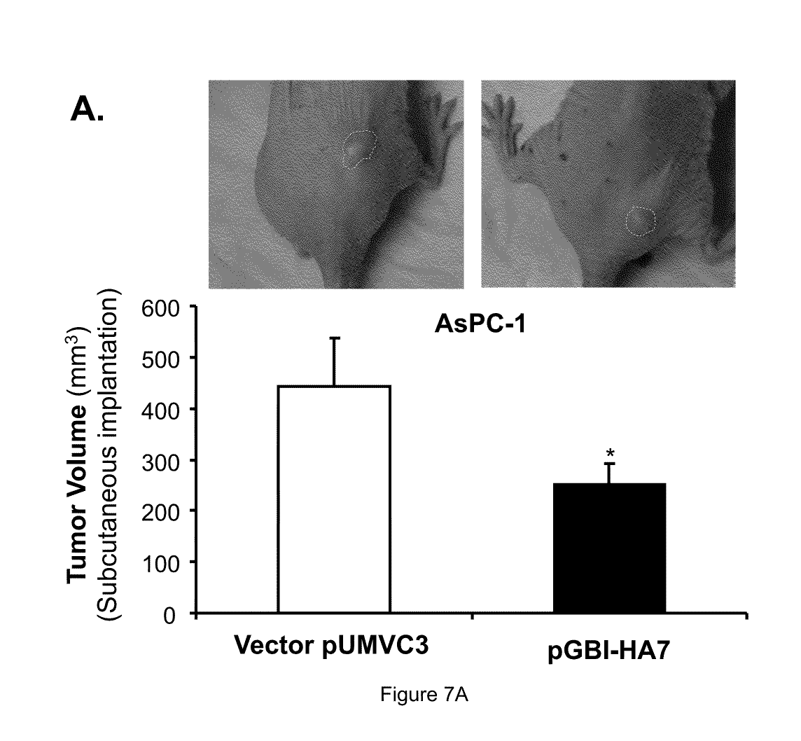

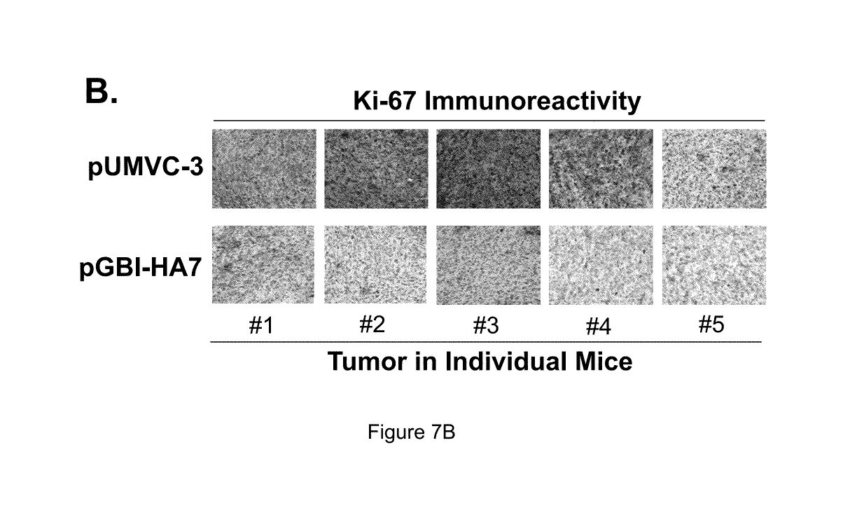

The effect of pGBI-HA7 on subcutaneous tumor growth. In order to determine whether antagomir pGBI-HA7 could inhibit pancreatic cancer growth in animal models, in this initial work we treated subcutaneous tumor with tail vein injection of either DNA-lipoplex pUMVC3 or pGBI-HA7 at 30 ug plasmid per injection for six treatments. As shown in FIG. 7A, the average tumor volume in pGBI-HA7 treated group was smaller significantly than that in the control group (p<0.05) at day 6 after the last treatment. In order to explore whether pGBI-HA7 could decrease proliferation of subcutaneous tumor, immunohistochemistry staining for ki-67, a molecular marker for proliferation, was performed. As showed in FIG. 7B and Table 2, pGBI-HA7 obviously reduced ki-67 staining compared with pUMVC-3. In agreement with the result of immunohistochemistry, the average mRNA level of ki-67 gene significantly decreased (p<0.05) in tumor samples harvested from the mice treated with pGBI-HA7 with respect to the control vector FIG. 7C). Furthermore, we also found a significantly decreased miR-196a expression level in tumor samples harvested from the mice treated with pGBI-HA7 compared with tumor samples from the mice treated with pUMVC-3 (FIG. 7D).

TABLE 2
ki-67 staining of subcutaneous tumors

| Number of mice | pUMVC-3* | pGBI-HA-7* |
|---|---|---|
| 1 | +2 | +1 |
| 2 | +3 | +1 |
| 3 | +3 | +1 |
| 4 | +3 | +1 |
| 5 | +2 | +2 |

*Staining score: 0, no staining; 1+, positive stain area <20% of cells; 2+, positive stain area in 20-50% of cells; +3, positive stain area >50% of cells.

Three plasmid-based miR-196a antagomirs were designed (pGBI-AS, pGBI-HA7, and pGBI-HB8). Among the three constructs pGBI-HA7, which expresses the miR-196a target sequence from 3′UTR of HOX-A7 mRNA, showed the best effects on decrease of miR-196a levels and inhibition of cell proliferation in PC cell line PANC-1. pGBI-HA7 also reduced PANC-1 and AsPC-1 cell proliferation and migration in the cell culture study, induced cell arrests at G1/G0 phase and increased p27^{Kip1} protein levels. PGBI-HA7 also inhibited tumor growth and reduced expression of a proliferation marker ki-67 in a subcutaneous tumor model. Thus, pGBI-HA7 might be a potential therapeutic for pancreatic cancer with high miR-196a expression.

Aberrantly upregulation of miR-196a has been reported to be implicated in progression of human beast, esophageal and colorectal cancers (6-8). We also demonstrated its higher expression in PC (5) and that specific knockdown of miR-196a in human pancreatic cancer cell lines inhibits tumor progression in vitro. All these data suggest overexpression of miR-196a may contribute to tumorigenesis and metastasis in these cancers. Thus, targeting miR-196a may be a new therapeutic strategy for these cancers. Antisense therapy for cancer is a form of treatment in which a synthesized strand of nucleic acids including DNA or RNA bind to the messenger RNA of a target gene, which is important for tumorigenesis or metastasis, and shut down its function, thereby achieving therapeutic effects (12). There are two approaches used for antisense oligonucleotide delivery: the first one is that small DNAs or RNAs are synthesized and then delivered into cells or the body (12); the second approach is to use a vector-base delivery system, such as plasmid or viral vector-mediated oligonucleotide delivery (13, 14). However, each of these methods has drawbacks. A major problem for oligonucleotide delivery is that it is hard to control the distribution of oligonucleotides once they have entered the body systemically, and thus the therapeutic oligonucleotides may not reach the target site efficiently; second, oligonucleotide RNAs are easily to get degredated and difficult to synthesize to a large amount for therapeutic purpose (15). The viral vector based delivery system has advantages of a high transduction efficiency. However, viral vector delivery system always raise concerns about their inducing immune response in the body and randomly inserted inactivation of a tumor suppressor gene or activation of an oncogene, which has potential to induce another type of cancer (16, 17). On the other hand, although plasmid-based delivery system does not induce strong immune response as a viral vector does, its transfection efficiency is relatively low compared with a viral vector (18).

In our three designed constructs for PC therapy, only pGBI-HA7 showed a knockdown effect on miR-196a levels while the other two vectors pGBI-AS and pGBI-HB8 did not. We do not know the exact reasons for this observation. However, among the three constructs, only pGBI-HA7 expresses a natural part sequence from 3′ UTR of HOX-A7, which contains five seed sequences for binding miR-196a and natural flaking region sequences, while the other two constructs pGBI-AS and pGBI-HB-8 contain one completely complementary sequence of miR-196a and five repeated miR-196a binding sequence from 3′ UTR of HOX-B8 mRNA, respectively. This observation may indicate that the flanking regions are also very important when we design an antisense to knockdown miRNA expression. Notably, although pGBI-AS and pGBI-HB8 did not decreased miR-196a levels, they still reduced PANC-1 cell proliferation. This suggests that pGBI-AS and pGBI-HB8 also work for functional inhibition of miR-196a, although the effect was less than that mediated by pGBI-HA7.

Since both HOX-A7 and HOX-B8 are experimentally validated targets of miR-196a (9), we checked their gene expression in PANC-1 and AsPC-1 cell lines by real-time PCR assay. However, mRNA of HOAX-A7 nor HOX-B8 was not detected in these cells (data not shown), which suggests these homeobox genes may not play roles in miR-196a-mediated signaling pathways in these PC cell lines. However, when a large quantity of miR-196a binding RNA copies are produced exogenously by our plasmid constructs, these antisense-like RNAs can bind to miR-196a and act as decoy binding sequences for miR-196a, thereby inhibiting its function.

miR-196a has been implicated in several cancers and the functional contributions of miR-196a to different types of cancers are quite different. In colorectal cancer, higher miR-196a expression seems to be associated with metastasis as a functional study shows that transient transfection of miR-196a into a colonal cancer cell line SW480 promotes cancer cell detachment, migration, invasion and chemosensitivity, but does not impact on proliferation or apoptosis (8). miR-196a also increases the development of lung metastases in mice after tail vein injection of transiently transfected SW480 cells (8). In a similar study, it is demonstrated that miR-196a promotes beast cancer and esophageal cancer cell proliferation, anchorage-independent growth and suppressed apoptosis (7). The above studies suggest that miR-196a has an oncogenic role in these cancers, which are consistent with our data in this study. However, miR-196a has been reported to exert a tumor suppression effect in other cancers. For example, miR-196a levels were reduced in melanoma cells compared to healthy melanocyte controls and reduced expression or functional inhibition of miR-196a in normal melanocytes increased cell migration, while re-expression of miR-196a in melanoma cells significantly inhibited cell invasion potential (19, 20). We believe that those inconsistent results regarding miR-196a expression and its functions reflect the complexity of miR-196a expression regulation and its target genes. Certain cellular molecules or pathways are likely to control miR-196a expression, which may explain why miR-196a is unregulated in cancer originated from colon, pancreas, or breast, while it is downregulated in melanoma cells. With respect to miR-196a function, selection of different target genes for miR-196a may play an important role in determining miR-196a function. For example, in melanoma, miR-196a exhibits its anti-tumor effects through downregulating oncogenes HOX-B7 and/or HOX-C8 (19). On the other hand, we observed that PANC-1 and AsPC-1 have no expression of these homeobox genes (data not shown) regardless of miR-196a expression levels, therefore, it is unlikely that these oncogenes play any roles in these PC cells. However, we find that tumor suppressor p27^{Kip1} gene is upregulated by miR-196a inhibition in these PC cell lines and that p27^{Kip1} gene is one of predicted target genes for miR-196a by searching relevant miRNA target prediction databases.

As for the efficacy of antagomir pGBI-HA7 on tumor growth in our initial subcutaneous mouse model, we did observe slower tumor growth and a decreased expression of proliferation marker ki-67 with pGBI-HA7 treatment. In addition, we also detected a decreased miR-196a expression level in tumor samples from mice treated with pGBI-HA7. This downregulation of miR-196a, we believe, is driven by pGBI-HA7 treatment, which indicates that pGBI-HA7 works for therapeutic purpose in this subcutaneous mouse model. Although the tumor growth difference was statistically significant between the treatment and control group, several factors could have negative impact on the efficacy result in this subcutaneous mouse model. One big limitation is limited blood flow, which carries the drugs, into the subcutaneous tumor when we introduce the therapeutics into tumor via tail vein injection, which could result in less therapeutics into target tumor sites. In order to overcome the shortage of this model, we will use intratumor injection of therapeutic plasmids in a subcutaneous tumor model or tail vein injection in an orthtopic pancreatic tumor model for our further study of pGBIHA7.

In conclusion, miR-196a antagomir pGBI-HA7 significantly reduces miR-196 expression and inhibits cell proliferation, cell migration and cell cycle progression in two human pancreatic cancer cell lines that highly express miR-196a. Mechanistically, pGBI-HA7 may play a decoy role to reduce functional levels of miR-196a, thereby increasing miR-196a targeting gene translation such as tumor suppressor gene p27. PGBI-HA7 also inhibited tumor growth and reduced expression of a proliferation marker ki-67 in a subcutaneous tumor model. These data suggest that miR-196a antagomir may have great potential as a novel and specific therapeutic agent for the treatment of human pancreatic cancer.

Listed Sequences

SEQ ID No: 1:
GTGCTGCGGCGAGCTCCGTCCAAAAGAAAATGGGGTTTGGTGTAAATCTGGGGGTG
TAATGTTATCATATATCACTCTACCTCGTAAAACCGACACTGAAAGCTGCCGGACAACAAA
TCACAGGTCAAAATTATGAGTTCTTCGTATTATGTGAACGCGCTTTTTAGCAAATATACGGC
GGGGGCTTCTCTGTTCCAAAATGCCGAGCCGACTTCTTGCTCCTTTGCTCCCAACTCACAGA
GAAGCGGCTACGGGGCGGGCGCCGGCGCCTTCGCCTCGACCGTTCCGGGCTTATACAATGT
CAACAGCCCCCTTTATCAGAGCCCCTTTGCGTCCGGCTACGGCCTGGGCGCCGACGCCTACG
GCAACCTGCCCTGCGCCTCCTACGACCAAAACATCCCCGGGCTCTGCAGTGACCTCGCCAA
AGGCGCCTGCGACAAGACGGACGAGGGCGCGCTGCATGGCGCGGCTGAGGCCAATTTCCG
CATCTACCCCTGGATGCGGTCTTCAGGACCTGACAGGAAGCGGGGCCGCCAGACCTACACG
CGCTACCAGACGCTGGAGCTGGAGAAGGAGTTCCACTTCAACCGCTACCTGACGCGGCGCC
GCCGCATTGAAATCGCCCACGCGCTCTGCCTCACCGAGCGCCAGATTAAGATCTGGTTCCA
GAACCGCCGCATGAAGTGGAAGAAAGAGCATAAGGACGAAGGTCCGACTGCCGCCGCAGC
TCCCGAGGGCGCCGTGCCCTCTGCCGCCGCCACTGCTGCCGCGGACAAGGCCGACGAGGAG
GACGATGATGAAGAAGAGGAAGACGAGGAGGAATGAGGGGCCGATCCGGGGCCCTCTCTG
CACCGGACAGTCGGAAAAGCGTCTTTAAGAGACTCACTGGTTTTACTTACAAAAATGGGAA
AAATAAAAGAAAATGTAAAAAACAAAAACAAAAACAAAAAAGCAACCCAGTCCCCAACCT
GCACTCTACCCACCCCCATCACCTACTCCAGCTCCCAACTTTTGTGGACTGAGCGGCCGCAG
AGACTGGGTCGCCTTGGATTCCCTCTGCCTCCGAGGACCCCAAAAGACACCCCCAACCCCA
GGCCAGCCGGCCCTGCTCTGGCGCGTCCAAAATACTACCTAGCACAGGCCTCTGCTCGAGG
CACCCCCAAACTACCTATGTATCCAGCCCCAGAGGGCCTCCATTCCCAGGAAGTCCCTATGT
ATCCCAACACTGGCAGACACCCAGCACCACCCTCCCAGACCCGCAAGAAAGTGAATCTCAC
TACTACCTACTCCCCTAAAACTACCTATTTTGTGCTGGCTGGCTTGCCTGCTACCTAGTGCCG
ACTGCTCCCAGGCAAGTCCCCTGCTGCTTACAGCCCGCAGCTTTTGGGGTCCCTGAGGCTGC
CCTGAGAATGTGCTGAGGTCCAGGATCAGGGTATTGGCATCTATTTAAATCGAAAAATAAT
ATATTTATTCCAAAAAGCATCCTAAGTGCTTGCACCCTAGAATCAATCCCTCCTTCTCTGGC
TTGGCACCCACAGCTCAGGCCCATCAACCCCCACTTCTGGAGGGGAATGTTCCTGAGCTGG
CTGCAGATCTGTGGGTTAGCTTCTGCTTAGCAGGACTGTGGAGATGCTTCCAGCTTCGCTGT
CCTTTCCTCTGGCTCCTGTATCTTACTGTTCAGCTGTGTTAAATATGTACGCCCTGATGTTTC
CTATAATAGCAGATACTGTATATTTGAACAAGATTTTTTTTTATCATTTCTATAGTCTTGGAG
TTCATTTGTAAGGCAGTGTCTTGACTTGGAAAGGATGTGTTAATGGGGTGACTTTGTAGCAT
GGTATGTTGTCTTGAGTTAACTGTAGTGGGTGGGGAGGTCCAATGCCCTCCGCAATGCCCTT
CATCTCCTGTGTTGTCCTGTACCCTGCTCAGCTCCATCCTGGGGTTCAGGGAAGGCACACTT
CCCAGCCCAGCTGTGTTTTATGTAACCGAAAATAAAGATGCGTGGTGACAAAGAAAAA
SEQ ID No: 2:
CCGGCCCTGCTCTGGCGCGTCCAAAATACTACCTAGCACAGGCCTCTGCTCGAGGCA
CCCCCAAACTACCTATGTATCCAGCCCCAGAGGGCCTCCATTCCCAGGAAGTCCCTATGTAT
CCCAACACTGGCAGACACCCAGCACCACCCTCCCAGACCCGCAAGAAAGTGAATCTCACTA
CTACCTACTCCCCTAAAACTACCTATTTTGTGCTGGCTGGCTTGCCTGCTACCTAGTGCCGAC
TGCTCCCA
SEQ ID No: 3:
CCGGCCCTGCTCTGGCGCGTCCAAAATACTACCTAGCACAGGCCTCTGCTCGAGGCA
CCCCCAAACTACCTTGTATCCAGCCCGACCCGCAAGAAAGTGAATCTCACTACTACCTCTCC
CCTAAAACTACCTTTTTGTGCTGGCTGGCTTGCCTGCTACCTAGTGCCGACTGCTCCCA
SEQ ID No: 4
TGGCCATTGCATACGTTGTATCCATATCATAATATGTACATTTATATTGGCTCATGTC
CAACATTACCGCCATGTTGACATTGATTATTGACTAGTTATTAATAGTAATCAATTACGGGG
TCATTAGTTCATAGCCCATATATGGAGTTCCGCGTTACATAACTTACGGTAAATGGCCCGCC
TGGCTGACCGCCCAACGACCCCCGCCCATTGACGTCAATAATGACGTATGTTCCCATAGTAA
CGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGTACATC
TACGTATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCA
AATGGGCGGTAGGCGTGTACGGTGGGAGGTCTATATAAGCAGAGCTCGTTTAGTGAACCGT
CAGATCGCCTGGAGACGCCATCCACGCTGTTTTGACCTCCATAGAAGACACCGGGACCGAT
CCAGCCTCCGCGGCCGGGAACGGTGCATTGGAACGCGGATTCCCCGTGCCAAGAGTGACGT
AAGTACCGCCTATAGACTCTATAGGCACACCCCTTTGGCTCTTATGCATGCTATACTGTTTTT
GGCTTGGGGCCTATACACCCCCGCTTCCTTATGCTATAGGTGATGGTATAGCTTAGCCTATA
GGTGTGGGTTATTGACCATTATTGACCACTCCAACGGTGGAGGGCAGTGTAGTCTGAGCAG
TACTCGTTGCTGCCGCGCGCGCCACCAGACATAATAGCTGACAGACTAACAGACTGTTCCTT
TCCATGGGTCTTTTCTGCAGTCACCGTCGTCGACCGGCCCTGCTCTGGCGCGTCCAAAATAC
TACCTAGCACAGGCCTCTGCTCGAGGCACCCCCAAACTACCTTGTATCCAGCCCGACCCGCA
AGAAAGTGAATCTCACTACTACCTCTCCCCTAAAACTACCTTTTTGTGCTGGCTGGCTTGCC
TGCTACCTAGTGCCGACTGCTCCCAGCGGCCGCGGATCCAGATCTTTTTCCCTCTGCCAAAA
ATTATGGGGACATCATGAAGCCCCTTGAGCATCTGACTTCTGGCTAATAAAGGAAATTTATT
TTCATTGCAATAGTGTGTTGGAATTTTTTGTGTCTCTCACTCGGAAGGACATATGGGAGGGC
AAATCATTTAAAACATCAGAATGAGTATTTGGTTTAGAGTTTGGCAACATATGCCCATTCTT
CCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCTGA
CGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTA
AACTTGGTCTGACAGTTACCAATGCTTAATCAGTGAGGCACCTATCTCAGCGATCTGTCTAT
TTCGTTCATCCATAGTTGCCTGACTCGGGGGGGGGGGGCGCTGAGGTCTGCCTCGTGAAGA
AGGTGTTGCTGACTCATACCAGGCCTGAATCGCCCCATCATCCAGCCAGAAAGTGAGGGAG
CCACGGTTGATGAGAGCTTTGTTGTAGGTGGACCAGTTGGTGATTTTGAACTTTTGCTTTGC
CACGGAACGGTCTGCGTTGTCGGGAAGATGCGTGATCTGATCCTTCAACTCAGCAAAAGTT
CGATTTATTCAACAAAGCCGCCGTCCCGTCAAGTCAGCGTAATGCTCTGCCAGTGTTACAAC
CAATTAACCAATTCTGATTAGAAAAACTCATCGAGCATCAAATGAAACTGCAATTTATTCAT
ATCAGGATTATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGACTCGTCCAA
CATCAATACAACCTATTAATTTCCCCTCGTCAAAAATAAGGTTATCAAGTGAGAAATCACCA
TGAGTGACGACTGAATCCGGTGAGAATGGCAAAAGCTTATGCATTTCTTTCCAGACTTGTTC
AACAGGCCAGCCATTACGCTCGTCATCAAAATCACTCGCATCAACCAAACCGTTATTCATTC
GTGATTGCGCCTGAGCGAGACGAAATACGCGATCGCTGTTAAAAGGACAATTACAAACAGG
AATCGAATGCAACCGGCGCAGGAACACTGCCAGCGCATCAACAATATTTTCACCTGAATCA
GGATATTCTTCTAATACCTGGAATGCTGTTTTCCCGGGGATCGCAGTGGTGAGTAACCATGC
ATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAA
CAACTCTGGCGCATCGGGCTTCCCATACAATCGATAGATTGTCGCACCTGATTGCCCGACAT
TATCGCGAGCCCATTTATACCCATATAAATCAGCATCCATGTTGGAATTTAATCGCGGCCTC
GAGCAAGACGTTTCCCGTTGAATATGGCTCATAACACCCCTTGTATTACTGTTTATGTAAGC
AGACAGTTTTATTGTTCATGATGATATATTTTTATCTTGTGCAATGTAACATCAGAGATTTTG
AGACACAACGTGGCTTTCCCCCCCCCCCCATTATTGAAGCATTTATCAGGGTTATTGTCTCA
TGAGCGGATACATATTTGAATGTATTTAGAAAAATAAACAAATAGGGGTTCCGCGCACATT
TCCCCGAAAAGTGCCACCTGACGTCTAAGAAACCATTATTATCATGACATTAACCTATAAA
AATAGGCGTATCACGAGGCCCTTTCGTCTCGCGCGTTTCGGTGATGACGGTGAAAACCTCTG
ACACATGCAGCTCCCGGAGACGGTCACAGCTTGTCTGTAAGCGGATGCCGGGAGCAGACAA
GCCCGTCAGGGCGCGTCAGCGGGTGTTGGCGGGTGTCGGGGCTGGCTTAACTATGCGGCAT
CAGAGCAGATTGTACTGAGAGTGCACCATATGCGGTGTGAAATACCGCACAGATGCGTAAG
GAGAAAATACCGCATCAGATTGGCTAT
SEQ ID No: 5
CGGCCCTGCTCTGGCGCGTCCAAAATACTACCTAGCACAGGCCTCTGCTCGAGGCAC
CCCCAAACTACCTTGTATCCAGCCCGACCCGCAAGAAAGTGAATCTCACTACTACCTCTCCC
CTAAAACTACCTTTTTGTGCTGGCTGGCTTGCCTGCTACCTAGTGCCGACTGCTCCCAGCGG
CCGCGGATCCA
SEQ ID No: 6:
CCCAACAACATGAAACTACCTA
SEQ ID No: 7:
CCCAACAACATGAAACTGCCTA
SEQ ID No: 8:
TCTCCCAACAACATGAAACTGCCTATTCAC
SEQ ID No: 9:
TCGACTGCTGTTGAAGTGAGCGCCTAGCATGTTTCATGTTGATCGGTAGTGAAGCCA
CAGATGTACCCAACAACATGAAACTACCTAGTTGCCTACTGCCTCGGAAGCTTAATAAAGG
ATCTTTTATTTTCATTGGC
SEQ ID No: 10:
TCTCCCAACAACATGAAACTGCCTATTCACAATTATTCTCCCAACAACATGAAACTG
CCTATTCACTCTCCCAACAACATGAAACTGCCTATTCACCAATATTCTCCCAACAACATGAA
ACTGCCTATTCAC
SEQ ID No: 11:
GGGUUGUUGUACUUUGAUGGAU

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1 Jemal, A., Siegel, R., Xu, J. and Ward, E. Cancer statistics, 2010, CA Cancer J. Clin., 60: 277-300, 2010.
2 Kim, E. J. and Simeone, D. M. Advances in pancreatic cancer, Curr. Opin. Gastroenterol., 27: 460-466, 2011.
3 Bhardwaj, A., Singh, S, and Singh, A. P. MicroRNA-based Cancer Therapeutics: Big Hope from Small RNAs, Mol. Cell Pharmacol., 2: 213-219, 2010.
4 Chan, E., Prado, D. E. and Weidhaas, J. B. Cancer microRNAs: from subtype profiling to predictors of response to therapy, Trends Mol. Med., 17: 235-243, 2011.
Zhang, Y., Li, M., Wang, H., Fisher, W. E., Lin, P. H., Yao, Q. and Chen, C. Profiling of 95 microRNAs in pancreatic cancer cell lines and surgical specimens by real-time PCR analysis, World J. Surg., 33: 698-709, 2009.
6 Luthra, R., Singh, R. R., Luthra, M. G., Li, Y. X., Hannah, C., Romans, A. M., Barkoh, B. A., Chen, S. S., Ensor, J., Maru, D. M., Broaddus, R. R., Rashid, A. and Albarracin, C. T. MicroRNA-196a targets annexin A1: a microRNA-mediated mechanism of annexin A1 downregulation in cancers, Oncogene, 27: 6667-6678, 2008.
7 Maru, D. M., Singh, R. R., Hannah, C., Albarracin, C. T., Li, Y. X., Abraham, R., Romans, A. M., Yao, H., Luthra, M. G., Anandasabapathy, S., Swisher, S. G., Hofstetter, W. L.,
Rashid, A. and Luthra, R. MicroRNA-196a is a potential marker of progression during Barrett's metaplasia-dysplasia-invasive adenocarcinoma sequence in esophagus, Am. J. Pathol., 174: 1940-1948, 2009.
8 Schimanski, C. C., Frerichs, K., Rahman, F., Berger, M., Lang, H., Galle, P. R., Moehler, M. and Gockel, I. High miR-196a levels promote the oncogenic phenotype of colorectal cancer cells, World J. Gastroenterol., 15: 2089-2096, 2009.
9 Yekta, S., Shih, I. H. and Bartel, D. P. MicroRNA-directed cleavage of HOXB8 mRNA, Science, 304: 594-596, 2004.
Zhang, L., Littlejohn, J. E., Cui, Y., Cao, X., Peddaboina, C. and Smythe, W. R. Characterization of bortezomib-adapted 1-45 mesothelioma cells, Mol. Cancer, 9: 110, 2010.
11 Larrea, M. D., Wander, S. A. and Slingerland, J. M. p27 as Jekyll and Hyde: regulation of cell cycle and cell motility, Cell Cycle, 8: 3455-3461, 2009.
12 Chan, J. H., Lim, S, and Wong, W. S. Antisense oligonucleotides: from design to therapeutic application, Clin. Exp. Pharmacol. Physiol, 33: 533-540, 2006.
13 Elsabahy, M., Nazarali, A. and Foldvari, M. Non-viral nucleic acid delivery: key challenges and future directions, Curr. Drug Deliv., 8: 235-244, 2011.
14 Lakka, S. S., Rajan, M., Gondi, C., Yanamandra, N., Chandrasekar, N., Jasti, S. L., Adachi, Y., Siddique, K., Gujrati, M., Olivero, W., Dinh, D. H., Kouraklis, G., Kyritsis, A. P. and Rao, J. S. Adenovirus-mediated expression of antisense MMP-9 in glioma cells inhibits tumor growth and invasion, Oncogene, 21: 8011-8019, 2002.
Seidl, C. I. and Ryan, K. Circular single-stranded synthetic DNA delivery vectors for microRNA, PLoS. One., 6: e16925, 2011.
16 Yi, Y., Hahm, S. H. and Lee, K. H. Retroviral gene therapy: safety issues and possible solutions, Curr. Gene Ther., 5: 25-35, 2005.
17 Yotnda, P., Chen, D. H., Chiu, W., Piedra, P. A., Davis, A., Templeton, N. S, and Brenner, M. K. Bilamellar cationic liposomes protect adenovectors from preexisting humoral immune responses, Mol. Ther., 5: 233-241, 2002.
18 Templeton, N. S., Lasic, D. D., Frederik, P. M., Strey, H. H., Roberts, D. D. and Pavlakis, G. N. Improved DNA: liposome complexes for increased systemic delivery and gene expression, Nat. Biotechnol., 15: 647-652, 1997.
19 Braig, S., Mueller, D. W., Rothhammer, T. and Bosserhoff, A. K. MicroRNA miR-196a is a central regulator of HOX-B7 and BMP4 expression in malignant melanoma, Cell Mol. Life Sci., 67: 3535-3548, 2010.
Mueller, D. W. and Bosserhoff, A. K. MicroRNA miR-196a controls melanoma-associated genes by regulating HOX-C8 expression, Int. J. Cancer, 129: 1064-1074, 2011.

What is claimed is:

1. An miR-196a antagonist capable of inhibiting a miR-196a activity, the miR-196a antagonist comprising one or more target sites for miR-196a.

2. The miR-196a antagonist of claim 1, comprising 1, 2, 3, 4, 5, 6, 7, 8, or 10 target sites for miR-196a.

3. The miR-196a antagonist of claim 1, comprising at least 11 target sites for miR-196a.

4. The miR-196a antagonist of claim 1, wherein the one or more target sites for miR-196a comprise one or more HOXA7 target site for miR-196a.

5. The miR-196a antagonist of claim 1, wherein the one or more target sites for miR-196a comprise at least five HOXA7 target site for miR-196a.

6. The miR-196a antagonist of claim 1, wherein the one or more target sites for miR-196a comprise one or more 3′ UTR of HOXB8 mRNA.

7. The miR-196a antagonist of claim 1, wherein the one or more target sites for miR-196a comprise one or more 3′ UTR of HOXB8 mRNA, wherein the one or more 3′ UTR of HOXB8 mRNA comprise at four miR-196a target sequences.

8. The miR-196a antagonist of claim 1, wherein the one or more target sites for miR-196a comprise at least 5 copies of 3′ UTR of HOXB8 mRNA.

9. The miR-196a antagonist of claim 1, wherein the one or more target sites for miR-196a comprise a sequence that is complementary to a mature miR-196a sequence.

10. The miR-196a antagonist of claim 1, wherein the one or more target sites for miR-196a comprise at least one stem-loop structure comprising a guide strand that comprises a sequence that is complementary to miR-196a, the stem-loop structure further comprising a passenger strand that comprises a mismatch.

11. The miR-196a antagonist of claim 1, wherein the one or more target sites for miR-196a comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof.

12. An expression vector comprising:
a promoter; and
a nucleic acid insert operably linked to the promoter,
wherein the insert encodes one or more miR-196a antagonists capable of inhibiting a miR-196a activity.

13. The expression vector of claim 12, selected from the group consisting of viral vector, lentiviral vector, and plasmid.

14. The expression vector of claim 12, wherein a vector backbone is miRZip or pUMVC3.

15. The expression vector of claim 12, wherein the vector is in a bilamellar invaginated vesicle (BIV) liposomal delivery system.

16. The expression vector of claim 12, wherein the vector is in a compacted DNA nanoparticle.

17. The expression vector of claim 12, wherein the vector is compacted with one or more polycations that is a 10 kDA polyethylene glycol (PEG)-substituted cysteine-lysine 3-mer peptide (CK₃₀PEG10k).

18. The expression vector of claim 12, wherein the vector is in a liposome comprising small molecule bivalent beta-turn mimics as receptor targeting moieties.

19. The expression vector of claim 12, wherein the one or more miR-196a antagonist comprise 1, 2, 3, 4, 5, 6, 7, 8, or 10 target sites for miR-196a.

20. The expression vector of claim 12, wherein the one or more miR-196a antagonists comprise at least 11 target sites for miR-196a.

21. The expression vector of claim 12, wherein the one or more miR-196a antagonists comprise one or more HOXA7 target site for miR-196a.

22. The expression vector of claim 12, wherein the one or more miR-196a antagonists comprise at least five HOXA7 target site for miR-196a.

23. The expression vector of claim 12, wherein the one or more miR-196a antagonists comprise one or more 3′ UTR of HOXB8 mRNA.

24. The expression vector of claim 12, wherein the one or more miR-196a antagonists comprise one or more 3′ UTR of HOXB8 mRNA, wherein the one or more 3′ UTR of HOXB8 mRNA comprise at four miR-196a target sequences.

25. The expression vector of claim 12, wherein the one or more miR-196a antagonists comprise comprises at least 5 copies of 3′ UTR of HOXB8 mRNA.

26. The expression vector of claim 12, wherein the one or more miR-196a antagonists comprise a sequence that is complementary to a mature miR-196a sequence.

27. The expression vector of claim 12, wherein the one or more miR-196a antagonists comprise at least one stem-loop structure comprising a guide strand that comprises a sequence that is complementary to miR-196a, the stem-loop structure further comprising a passenger strand that comprises a mismatch.

28. The expression vector of claim 12, wherein the one or more miR-196a antagonists comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof.

29. A method for suppressing tumor cell growth, treating pancreatic ductal adenocarcinoma, or both in a human subject comprising the steps of:
identifying the human subject in need for suppression of the tumor cell growth, treatment of pancreatic ductal adenocarcinoma or both; and
administering an expression vector in a therapeutic agent carrier complex to the human subject in an amount sufficient to suppress the tumor cell growth, treat pancreatic ductal adenocarcinoma or both, wherein
the expression vector encodes one or more miR-196a antagonists capable of inhibiting a miR-196a activity in one or more target cells, wherein the inhibition results in suppressed tumor growth, a reduced tumor cell proliferation, or a reduced invasiveness of the tumor cells.

30. The method of claim 29, wherein the therapeutic agent carrier is a compacted DNA nanoparticle or a reversibly masked liposome decorated with one or more “smart” receptor targeting moieties that are small molecule bivalent beta-turn mimics.

31. The method of claim 29, wherein the therapeutic agent carrier is compacted DNA nanoparticles that are further encapsulated in a liposome.

32. The method of claim 29, wherein the therapeutic agent carrier is a compacted DNA nanoparticle compacted with one or more polycations, wherein the one or more polycations is a 10 kDA polyethylene glycol (PEG)-substituted cysteine-lysine 3-mer peptide (CK30PEG10k) or a 30-mer lysine condensing peptide.

33. The method of claim 29, wherein the therapeutic agent carrier comprises reversibly masked liposome that are bilamellar invaginated vesicle (BIV).

34. The method of claim 29, wherein administering the vector comprises administering the vector before, after, or concurrently as a combination therapy with one or more treatment methods selected from the group consisting of chemotherapy, radiation therapy, surgical intervention, antibody therapy, Vitamin therapy, or any combinations thereof.

35. The method of claim 29, wherein the expression vector is selected from the group consisting of viral vector, lentiviral vector, and plasmid.

36. The method of claim 29, wherein the expression vector comprises a vector backbone that is miRZip or pUMVC3.

37. The method of claim 29, wherein the one or more miR-196a antagonists comprise 1, 2, 3, 4, 5, 6, 7, 8, or 10 target sites for miR-196a.

38. The method of claim 29, wherein the one or more miR-196a antagonists comprise at least 11 target sites for miR-196a.

39. The method of claim 29, wherein the one or more miR-196a antagonists comprise one or more HOXA7 target site for miR-196a.

40. The method of claim 29, wherein the one or more miR-196a antagonists comprise at least five HOXA7 target site for miR-196a.

41. The method of claim 29, wherein the one or more miR-196a antagonists comprise one or more 3′ UTR of HOXB8 mRNA.

42. The method of claim 29, wherein the one or more miR-196a antagonists comprise one or more 3′ UTR of HOXB8 mRNA, wherein the one or more 3′ UTR of HOXB8 mRNA comprise at four miR-196a target sequences.

43. The method of claim 29, wherein the one or more miR-196a antagonists comprise comprises at least 5 copies of 3′ UTR of HOXB8 mRNA.

44. The method of claim 29, wherein the one or more miR-196a antagonists comprise a sequence that is complementary to a mature miR-196a sequence.

45. The method of claim 29, wherein the one or more miR-196a antagonists comprise at least one stem-loop structure comprising a guide strand that comprises a sequence that is complementary to miR-196a, the stem-loop structure further comprising a passenger strand that comprises a mismatch.

46. The method of claim 29, wherein the one or more miR-196a antagonists comprise one or more sequences selected from the group consisting of SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, and combinations thereof.

47. A method of treating pancreatic ductal adenocarcinoma, or increasing effectiveness of a chemotherapeutic regimen to treat pancreatic ductal adenocarcinoma, or both in a human or animal subject, comprising the steps of:
identifying the human or animal subject suffering from pancreatic ductal adenocarcinoma or needing increased effectiveness of the chemotherapy against pancreatic ductal adenocarcinoma; and
administering an expression vector in a therapeutic agent carrier complex to the human or animal subject in an amount sufficient to suppress or inhibit miR-196a activity in the human or the animal subject, wherein the expression vector expresses one or more miR-196a antagonists capable of inhibiting a miR-196a activity in one or more target cells in the human or animal subject, wherein the inhibition results in an enhanced action of the one or more chemotherapeutic agents, an arrested proliferation, reduced proliferation, or a reduced invasiveness of one or more tumor cells.
